(19) United States
(12) Patent Application Publication
(10) Pub. No.: US 2026/0201351 A1
(43) Pub. Date: Jul. 16, 2026

(54) MUTANT TRANSAMINASE WITH INCREASED ASYMMETRIC REDUCTIVE AMINATION ACTIVITY AS WELL AS METHODS AND USES INVOLVING THE SAME

(71) Applicant: Hoffmann-La Roche Inc., Little Falls, NJ (US)

(72) Inventors: Rebecca Maria Ursula Buller, Unterengstringen (CH); Nadine Nina Duss, Stäfa (CH); Sumire Honda Malca, Wädenswil (CH); Hans Iding, Magden (CH); Gabi Daniela Milbredt, Wädenswil (CH); Michael Niklaus, Schmerikon (CH); Patrick Franz Stocker, Basel (CH)

(21) Appl. No.: 19/532,787

(22) Filed: Feb. 6, 2026

Related U.S. Application Data
(63) Continuation of application No. PCT/EP2024/068497, filed on Jul. 1, 2024.

(30) Foreign Application Priority Data
Aug. 9, 2023 (EP) 23190650.4

(51) Int. Cl. C12N 9/10; C12N 1/20; C12P 13/00; C12R 1/19

(52) U.S. Cl. CPC C12N 9/1096; C12N 1/20; C12P 13/001; C12R 2001/19; C12Y 206/01

(57) ABSTRACT

The present invention relates to a mutant transaminase, a nucleic acid encoding the mutant transaminase, a vector comprising the nucleic acid, a cell comprising the mutant transaminase or nucleic acid and a method for the enzymatic reductive transamination of a ketone and the formation of a primary amine in the presence of mutant transaminase.

### CROSS-REFERENCE TO RELATED APPLICATIONS

This application is a continuation of International PCT Application No. PCT/EP2024/068497 filed on Jul. 1, 2024, which claims priority to European Patent Application No. 23190650.4 filed on Aug. 9, 2023, the disclosures of which are hereby incorporated by reference in their entirety.

### SEQUENCE LISTING

This application incorporates by reference in its entirety the material in the ST.26 XML file titled ROCHE-265 Sequence Listing.xml, which was created on Jan. 28, 2026 and is 23,256 bytes.

The present invention relates to a mutant transaminase, a nucleic acid encoding the mutant transaminase, a vector comprising the nucleic acid, a cell comprising the mutant transaminase or nucleic acid and a method for the enzymatic reductive transamination of a ketone and the formation of a primary amine in the presence of mutant transaminase.

Transaminases (also referred to as aminotransferases) catalyze a transamination reaction, i.e. the transfer of an amino group from an amine donor to an amine acceptor, particularly the amination of a ketone along with the deamination of an amine, wherein the NH₂ group on one molecule or domain is exchanged with the carbonyl group on the other molecule or domain. Amine transaminases (ATAs) are ω-transaminases that catalyze the intermolecular transfer of an amine group between an amine donor and a carbonyl group-containing amine acceptor (i.e., a ketone or an aldehyde). ATAs use pyridoxal-5′-phosphate (PLP) as a cofactor and the mechanism involves oxidative deamination and reductive amination half reactions with PLP acting as the amine shuttle. Because of the reversibility of the transaminase reaction, ATAs can be applied in the preparation of optically pure amines using two approaches: (1) kinetic resolution of racemic amines via oxidative deamination, in which one enantiomer is converted into the corresponding ketone, leaving the desired amine enantiomer untouched, or (2) asymmetric synthesis of primary amines via reductive amination of prochiral ketones. Common amine donors used in the reductive amination reaction are alanine, 1-phenylethylamine (1-PEA) and isopropylamine (2-PrNH₂). The latter is often preferred for industrial purposes, as 2-PrNH₂ is achiral, inexpensive and the equilibrium can be driven by using excess 2-PrNH₂ or by removing the formed acetone byproduct under low pressure or slight heating.

During the last decade several(S)- and (R)-selective ATAs have been investigated and engineered for the efficient synthesis of high-value chiral amines in the pharmaceutical and agrochemical industries.

For the synthesis of chiral amines in the pharmaceutical industry, it was intended to develop through enzyme engineering, mutants with increased reductive amination activity and stereoselectivity towards a single enantiomer. In addition, stability in the presence of an organic solvent were desirable, because these traits would respectively allow removal of by-products by evaporation and application of the reaction in the presence of high concentration of an organic solvent or in an organic solvent. Further, it was to favor the formation of the amine product by shifting equilibrium. This could be attained by carrying out the reaction in an organic solvent. To account for the latter approach, it was intended to develop mutants with high performance in an organic solvent.

Surprisingly, it has been found that a mutant transaminase comprising an amino acid sequence that is at least 80% identical to the amino acid sequence of SEQ ID NO: 1 (transaminase from Ruegeria pomeroyi), wherein the mutant transaminase has at least four amino acid substitutions at positions 61, 65, 266 and 419 relative to the amino acid sequence of SEQ ID NO: 1, and wherein at least three of the substitutions are selected from the group consisting of:
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266); and
the amino acid at the position corresponding to position 419 of SEQ ID NO: 1 is substituted with Pro (Pro419) or Val (Val419)
   shows an increased asymmetric reductive amination activity relative to the wild-type transaminase, particularly the transaminase of Ruegeria pomeroyi, especially that of SEQ ID NO: 1 (see below).

As shown in the Examples, a core motif of mutations has been identified, which increases the asymmetric reductive amination activity of mutant transaminases. It has been found that mutant transaminases having at least four amino acid substitutions at positions 61, 65, 266 and 419 relative to the amino acid sequence of SEQ ID NO: 1 wherein at least three of the substitutions are selected from the group consisting of:
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266); and
the amino acid at the position corresponding to position 419 of SEQ ID NO: 1 is substituted with Pro (Pro419) or Val (Val419)
   show an increased asymmetric reductive amination activity relative to the wild-type transaminase, particularly the transaminase of Ruegeria pomeroyi, especially that of SEQ ID NO: 1. A single substitution at positions 61 (e.g. to Leu), 65 (e.g. to Gly, Ser, Ala, Met, Leu, Phe, Val or Cys), 266 and 419 (e.g. to Leu, Val, Pro, Ala, Cys or Gly) provided a minor increase in activity (see Tables 4, 6, 12 and 17), whereas the combination of four substitutions at the above positions provided a major increase in activity (see e.g. Tables 5, 7, 13 and 18). Additional mutations in the transaminase can further increase enzymatic activity (e.g. at positions 9, 62, 171, 198, 318, 320, 420, 421 and 464 (see Tables in section “Examples”, particularly Tables 2, 3, 9, 11, and 16).

Accordingly, in a first aspect the present invention relates to a mutant transaminase with increased asymmetric reductive amination activity relative to the wild-type transaminase, wherein the mutant transaminase comprises an amino acid sequence that is at least 80% identical to the amino acid sequence of SEQ ID NO: 1 (transaminase from Ruegeria pomeroyi),

wherein the mutant transaminase has at least four amino acid substitutions at positions 61, 65, 266 and 419 relative to the amino acid sequence of SEQ ID NO: 1, and
wherein at least three of the substitutions are selected from the group consisting of:
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266); and
the amino acid at the position corresponding to position 419 of SEQ ID NO: 1 is substituted with Pro (Pro419) or Val (Val419).

The term “transaminase” (classified as EC 2.6.1.XX by the Enzyme Commission of the International Union of Biochemistry; known also as aminotransferases) generally means an enzyme that catalyses the transfer of an amine group from an amine donor to the carbonyl group of an amine acceptor (transamination). Transaminases are pyridoxal-5′-phosphate dependent (PLP-dependent) enzymes. The amine donor provides the amino group to the amine acceptor-so that the desired amine is synthesized- and a corresponding ketone is formed. As transaminases often possess a high stereoselectivity, the transamination reaction can provide the desired amine by asymmetric reduction and/or the remaining amine donor by resolution via oxidative deamination as enantiomerically enriched amine. ATAs are of particular interest in the present invention, as they are able to convert ketones into chiral amines. In accordance with the present invention, the mutant transaminase is preferably active as ATA. This means that the mutant transaminase is capable of intermolecularly transferring an amine group from an amine donor to a carbonyl group-containing amine acceptor (i.e., a ketone or an aldehyde), under suitable conditions, as detailed above and below.

The term “wild-type transaminase” as used herein means any transaminase which occurs as such in nature and which has not been mutated. The term “mutant transaminase” as used herein means any transaminase, which originates from a corresponding wild-type transaminase and in comparison to such wild-type transaminase has been amended in its amino acid sequence. For example, this may comprise the introduction, deletion, substitution or post-translational mutation of one or more amino acids at one or more positions. Preferably, the mutant transaminase differs from the wild-type transaminase by amino acid substitutions. Methods for creating mutations, such as amino acid substitutions, in amino acid sequences are well-known to the person skilled in the art. For example, such mutations may already be introduced on nucleic acid level leading to the expression of the desired mutated amino acid sequence. Suitable methods therefore are well-known to the person skilled in the art and partly also described below, e.g. in the context of nucleic acids according to the second aspect of the invention.

A suitable mutant transaminase according to the first aspect may originate from the wild-type transaminase of any organism. A preferred source is Ruegeria sp., especially Ruegeria pomeroyi also referred to as Silicibacter pomeroyi. Accordingly, especially preferred is the Ruegeria pomeroyi transaminase (PDB-code: 3HMU; see below SEQ ID NO: 1).

As detailed above, the transaminase of the invention has increased asymmetric reductive amination activity relative to the wild-type transaminase. Reductive amination, or the conversion of a carbonyl group to an amine via an iminium intermediate, is one of the most important reactions for synthesising chiral amines, a functional group that features in a considerable proportion of small biologically active molecules. Research is being directed towards the development of asymmetric processes for reductive amination as, in many cases, the stereocentre bearing the amine is crucial in determining its biological activity.

Increased asymmetric reductive amination activity relative to the respective wild-type transaminase without mutation means that the mutant has an increased reductive amination activity relative to the wild-type and that the mutant is stereoselective, i.e. it has a preference for the formation of one stereoisomer over the other. (S)-selective ATAs have been already known since two decades. Preferably, the transaminase is therefore an amine transaminase (ATA). Accordingly, the transaminase of the present invention is stereoselective with respect to the stereocentre bearing the amine, such as(S)-selective or (R)-selective, preferably(S)-selective. Stereoselectivity can be partial, where the formation of one stereoisomer is favored over the other, or it may be complete where only one stereoisomer is formed. Enantioselectivity is commonly reported in the art (typically as a percentage) as the enantiomeric excess (e.e.) calculated therefrom according to the formula [major enantiomer−minor enantiomer]/[major enantiomer+minor enantiomer]. Alternatively, the enantiomeric ratio or er (S:R) may be used to characterize stereoselectivity. The enantiomeric ratio is the ratio of the percent of one enantiomer (e.g. the(S)-enantiomer) in a mixture of enantiomers to that of the other enantiomer (e.g. the (R)-enantiomer).

As detailed above, the mutant transaminase according to the first aspect shows increased asymmetric reductive amination activity relative to the wild-type transaminase. Methods for determining transaminase activity are well-known in the art and described herein. Exemplary methods are also described in the Examples. Enzymatic conversion is a measure of the activity of enzyme. The activity may be determined in an enzyme assay measuring either the consumption of substrate or formation of product over time. A large number of different methods of measuring the concentrations of substrates and products exist and many enzymes can be assayed in several different ways as known to the person skilled in the art. To determine, whether a mutant transaminase according to the first aspect shows increased transaminase activity relative to the wild-type transaminase, transaminase activity of both transaminases is measured using the same method. The conditions when measuring the activity are usually standardized. In accordance with the present invention one may take a temperature of between 40° C. and 70° C., e.g. 50° C., 60° C. or 70° C. (as in the Examples) and a suitable pH value, such as pH 7) and substrate concentration. Tests for stability in the presence of an organic solvent include the presence of organic solvents, e.g. dimethyl sulfoxide (e.g. between 20% and 40% in water), isopropyl acetate or any of the organic solvents used in the Examples. Suitable organic solvents are ethers, esters or hydrocarbons. Particular ethers are diethoxymethan or t.butyl methylether. Particular hydrocarbon include toluene and particular esters include lower alkyl esters of acetic acid or propionic acid, such as ethyl acetate, isopropyl acetate, isobutyl acetate, iso-propyl 2-methylpropanoate or tert-butyl 2,2-dimethylpropanoate. Preferred are the named esters with more particular preference for isopropyl acetate.

For example, methods of determining enzymatic activity of a transaminase in general may be based on a fluorescence or colorimetric assay. HPLC-UV may be employed as well. Assays are usually performed under well controlled conditions including e.g. pH value, temperature, salt, buffers, and substrate concentration. Further, methods of determining enzymatic activity of a transaminase in general may comprise the detection of the concentration of product being formed or the educt consumed.

A mutant transaminase according to the first aspect showing increased transaminase activity relative to the wild-type transaminase, for example, shows an increase in transaminase activity by more than the onefold. The person skilled in the art knows statistical procedures to assess whether or not one value of enzyme activity is increased relative to another, such as Student's t-test or chi-square test. It is evident for the skilled person that any background signal has to be subtracted when analyzing the data.

The mutant transaminase of the present invention comprises an amino acid sequence that is at least 80% identical to the amino acid sequence of SEQ ID NO: 1 (transaminase from Ruegeria pomeroyi). The term “at least 80% identical” or “at least 80% sequence identity” as used herein means that the sequence of the mutant transaminase according to the present invention has an amino acid sequence characterized in that, within a stretch of 100 amino acids, at least 80 amino acids residues are identical to the sequence of the corresponding wild-type sequence. Sequence identity according to the present invention can, e.g., be determined by methods of sequence alignment in form of sequence comparison. Methods of sequence alignment are well known in the art and include various programs and alignment algorithms which have been described in, e.g., Pearson and Lipman (1988). Moreover, the NCBI Basic Local Alignment Search Tool (BLAST) is available from several sources, including the National Center for Biotechnology Information (NCBI, Bethesda, MD) and on the internet, for use in connection with the sequence analysis programs blastp, blastn, blastx, tbiastn and tbiastx. Percentage of identity of mutants according to the present invention relative to the amino acid sequence of SEQ ID NO: 1 is typically characterized using the NCBI Blast blastp with standard settings. Alternatively, sequence identity may be determined using the software GENEious with standard settings. In the present invention, alignment results presented are derived from the Software Geneious (version R8), using the global alignment protocol with free end gaps as alignment type, and Blosum62 as a cost matrix. In accordance with the present invention, the mutant transaminase has at least four amino acid substitutions relative to the wild-type transaminase, wherein the amino acids at the position corresponding to positions 61, 65, 266 and 419 are substituted.

The mutant transaminase of the first aspect of the present invention has at least three of the substitutions selected from the group consisting of:
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266); and
the amino acid at the position corresponding to position 419 of SEQ ID NO: 1 is substituted with Pro (Pro419) or Val (Val419).

The positions of mutations are identified in the present invention with reference to SEQ ID NO: 1, i.e. the amino acid sequence of the transaminase of Ruegeria pomeroyi. The corresponding mutation sites of transaminases other than that of SEQ ID NO: 1 can be identified by performing an amino acid alignment as detailed above (e.g. by using BLAST; Basic Local Alignment Search Tool available at http://blast.ncbi.nlm.nih.gov/Blast.cgi?PROGRAM=blastp&PAGE_TYPE=BlastSearch&LINK_LOC=blasthome with standard settings) or by comparison of the structures, if available, and identifying the corresponding amino acid. An example of a different wild-type transaminase is 3HMU (ATCC 700808) and the positions corresponding to positions 61, 65, 266 and 419 of SEQ ID NO:1 are: 59, 63, 264 and 417, respectively.

In preferred embodiments of the present invention, the mutant transaminase comprises or consists of an amino acid sequence that is at least 85%, 90%, 95%, 96%, 97%, 98%, or 99% identical to the amino acid sequence of SEQ ID NO: 1.

Particularly, the mutant transaminase has at least four amino acid substitutions at positions 61, 65, 266 and 419 relative to the amino acid sequence of SEQ ID NO: 1, wherein at least three of the substitutions are selected from the group consisting of:
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266); and
the amino acid at the position corresponding to position 419 of SEQ ID NO: 1 is substituted with Pro (Pro419) or Val (Val419).

In one embodiment of the present invention, the sequence of the mutant transaminase according to the present invention may comprise, in addition to the substitutions specified herein a combination of one or more deletion(s), substitution(s) or addition(s) as defined above.

In one embodiment, the sequence of the mutant transaminase according to the present invention may comprise, in addition to the substitutions specified herein one or more additional amino acid substitution(s), particularly one or more conservative amino acid substitutions. “Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

In one embodiment of the present invention, the mutant transaminase according to the present invention may comprise one or more amino acid addition(s), particularly small (e.g. up to 10 amino acids) C- or N-terminal amino acid additions.

Moreover, the transaminase according to the present invention may be part of a larger molecule, such as a fusion protein or may be immobilized on a solid support. Fusion proteins are proteins created by joining of two or more originally separate proteins. Accordingly, depending on the intended use of the transaminase it may be combined with a further protein into a fusion protein. The proteins may be fused via a linker or spacer, which increases the likelihood that that the proteins fold independently and behave as expected. Especially in the case where the linkers enable protein purification, linkers in protein fusions are sometimes engineered with cleavage sites for proteases or chemical agents that enable the liberation of the two separate proteins. Preferably, the fusion protein of the present invention comprises a tag, e.g. in order to ease purification, to assist in the proper folding in proteins, to prevent precipitation of the protein or to alter chromatographic properties. Additionally, or alternatively, the mutant may be immobilized on a solid support. The solid supports can be resin, glasses, metal or nanomaterials based on the application. The immobilization techniques rely on effective protein bioconjugation chemistries and are known to the person skilled in the art.

In addition to an increased transaminase activity, the mutant transaminase according to the first aspect may further relative to the wild-type transaminase have an increased stability in the presence of an organic solvent.

The term “increased stability in the presence of an organic solvent” relative to the wildtype transaminase means that the mutant transaminase is less prone to loss of (enzyme) activity in the presence of an organic solvent. In the presence of an organic solvent in the present invention means that the amount of the organic solvent is rather high, i.e. at least 20%, 30%, 40%, 50%, 60%, 70%, 80%, 90% or 95%. Please note that a residual amount of water is present in general in order to allow the formation of a hydration shell around the enzyme. This interaction of the enzyme surface with the surrounding water is fundamental to the activity of the enzyme. This residual amount of water will typically between 1.0% wt. and 5.0% wt., particularly between 1.4% wt. to 3.4% wt., more particularly between 1.8% wt. to 2.8% wt. of the total amount of the solvent. Please note that in the present invention, the solvent is preferably monophasic. The organic solvent be any of those tested in the Examples, such as dimethyl sulfoxide (e.g. between 20% and 40% in water), ethers, esters or hydrocarbons. Particular ethers are diethoxymethan or t.butyl methylether. Particular hydrocarbon include toluene and particular esters include lower alkyl esters of acetic acid or propionic acid, such as ethyl acetate, isopropyl acetate, isobutyl acetate, iso-propyl 2-methylpropanoate or tert-butyl 2,2-dimethylpropanoate. Preferred are the named esters with more particular preference for isopropyl acetate.

In a preferred embodiment of the present invention, the mutant transaminase of the first aspect is characterized in that
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266), and
the amino acid at the position corresponding to position 419 of SEQ ID NO: 1 is substituted with Pro (Pro419) or Val (Val419).

Particularly, the mutant transaminase of the first aspect is characterized in that
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266), and
the amino acid at the position corresponding to position 419 of SEQ ID NO: 1 is substituted with Pro (Pro419);

### OR

the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266), and
the amino acid at the position corresponding to position 419 of SEQ ID NO: 1 is substituted with Val (Val419).

It has been found that mutants having the above mutations are particularly suitable and show the desired properties (see Examples).

Accordingly, they were selected as basis for the identification of further optimized mutants, in which additional mutations were introduced.

One mutation which has proven to provide additional beneficial effects (especially for a mutant characterized by mutations Leu61, Met65, Val266 and Val419) is the substitution of the amino acid at the position corresponding to position 426 of SEQ ID NO: 1 with Val (Val426). Hence, in a preferred embodiment, the mutant transaminase of the present invention is characterized in the amino acid at the position corresponding to position 426 of SEQ ID NO: is substituted with Val (Val426).

Accordingly, the mutant transaminase of the first aspect may be characterized in that
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266);
the amino acid at the position corresponding to position 419 of SEQ ID NO: 1 is substituted with Pro (Pro419); and
the amino acid at the position corresponding to position 426 of SEQ ID NO: is substituted with Val (Val426)

### OR

the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266);
the amino acid at the position corresponding to position 419 of SEQ ID NO: 1 is substituted with Val (Val419); and
the amino acid at the position corresponding to position 426 of SEQ ID NO: is substituted with Val (Val426).

However and preferably, the mutant transaminase of the first aspect may be characterized in that
the amino acid at the position corresponding to position 61 of SEQ ID NO: 1 is substituted with Leu (Leu61); and
the amino acid at the position corresponding to position 65 of SEQ ID NO: 1 is substituted with Met (Met65); and
the amino acid at the position corresponding to position 266 of SEQ ID NO: 1 is substituted with Val (Val266); and
the amino acid at the position corresponding to position 419 of SEQ ID NO: 1 is substituted with Pro (Pro419);

### OR

the amino acid at the position corresponding to position 61 of SEQ ID NO: 1 is substituted with Leu (Leu61); and
the amino acid at the position corresponding to position 65 of SEQ ID NO: 1 is substituted with Met (Met65); and
the amino acid at the position corresponding to position 266 of SEQ ID NO: 1 is substituted with Val (Val266); and
the amino acid at the position corresponding to position 419 of SEQ ID NO: 1 is substituted with Val (Val419); and
the amino acid at the position corresponding to position 426 of SEQ ID NO: 1 is substituted with Val (Val426).

The main characteristic of the two above mutants (Leu61/Met65/Val266/Pro419 and Leu61/Met65/Val266/Val419/Val426) is not only their increased activity in the aqueous reactions. These mutants display activity in the presence of organic solvents, which is essentially absent in the wild-type and many other investigated mutants not possessing this set of mutations.

In the mutants of the present invention—as defined above—additional substitutions have been introduced and found beneficial—alone or in combination with each other. These include the following:
the amino acid at the position corresponding to position 9 of SEQ ID NO: 1 is substituted with Tyr (Tyr9); and/or
the amino acid at the position corresponding to position 62 of SEQ ID NO: 1 is substituted with Ser (Ser62); and/or
the amino acid at the position corresponding to position 65 of SEQ ID NO: 1 is substituted with Phe (Phe65), Gly (Gly65) or Met (Met65), particularly Met (Met65); and/or
the amino acid at the position corresponding to position 171 of SEQ ID NO: 1 is substituted with Phe (Phe171) or Trp (Trp171), and/or
the amino acid at the position corresponding to position 198 of SEQ ID NO: 1 is substituted with Leu (Leu198) or Met (Met198), particularly Leu (Leu198); and/or
the amino acid at the position corresponding to position 318 of SEQ ID NO: 1 is substituted with Lys (Lys318), and/or
the amino acid at the position corresponding to position 320 of SEQ ID NO: 1 is substituted with Cys (Cys320), Leu (Leu318), Lys (Lys320), or Met (Met320), particularly Met (Met320), and/or
the amino acid at the position corresponding to position 419 of SEQ ID NO: 1 is substituted with Ala (Ala419), Cys (Cys419), Gly (Gly419), Pro (Pro419) or Val (Val419), particularly Pro (Pro419) or Val (Val419), and/or
the amino acid at the position corresponding to position 420 of SEQ ID NO: 1 is substituted with Asn (Asn420), Asp (Asp420), Cys (Cys420) or Ser (Ser420), and/or
the amino acid at the position corresponding to position 421 of SEQ ID NO: 1 is substituted with Ser (Ser421), and/or
the amino acid at the position corresponding to position 464 of SEQ ID NO: is substituted with Met (Met464).

Further mutations may or may not be present.

Particularly, the mutant transaminase is characterized as follows:
the mutant transaminase has the substitutions Leu61, Met65, Val266 and Pro419; or
the mutant transaminase has the substitutions Leu61, Met65, Val266, Val419 and Val426; or
the mutant transaminase has the substitutions Leu61, Ser62, Met65, Phe171, Val266, Lys318, Pro419, Asn420 and Ser421; or
the mutant transaminase has the substitutions Leu61, Ser62, Met65, Phe171, Val266, Lys318, Met320, Pro419, Asn420 and Ser421; or
the mutant transaminase has the substitutions Leu61, Met65, Phe171, Val266, Cys320, Val419, Asn420, Ser421 and Val426; or
the mutant transaminase has the substitutions Tyr9, Leu61, Ser62, Met65, Phe171, Val266, Lys318, Pro419, Asn420 and Ser421; or
the mutant transaminase has the substitutions Tyr9, Leu61, Ser62, Met65, Phe171, Val266, Lys318, Met320, Pro419, Asn420 and Ser421; or
the mutant transaminase has the substitutions Tyr9, Leu61, Met65, Phe171, Val266, Cys320, Val419, Asn420, Ser421 and Val426; or
the mutant transaminase has the substitutions Leu61, Ser62, Met65, Phe171, Asp198, Val266, Lys318, Pro419, Asn420, Ser421 and Met464; or
the mutant transaminase has the substitutions Leu61, Ser62, Met65, Phe171, Asp198, Val266, Lys318, Met320, Pro419, Asn420, Ser421 and Met464; or the mutant transaminase has the substitutions Leu61, Met65, Phe171, Asp198, Val266, Cys320, Val419, Asn420, Ser421, Val426 and Met464.

More particularly, the mutant transaminase is characterized as follows:
the mutant transaminase has the substitutions Leu61, Met65, Val266 and Pro419 and wherein the amino acids at positions corresponding to positions 9, 62, 171, 198, 318, 320, 420, 421, 426 and 464 of SEQ ID No:1 are unsubstituted;
the mutant transaminase has the substitutions Leu61, Met65, Val266, Val419 and Val426 and wherein the amino acids at positions corresponding to positions 9, 62, 171, 198, 318, 320, 420, 421 and 464 of SEQ ID No:1 are unsubstituted; or
the mutant transaminase has the substitutions Leu61, Ser62, Met65, Phe171, Val266, Lys318, Pro419, Asn420 and Ser421 and wherein the amino acids at positions corresponding to positions 9, 198, 320, 426 and 464 of SEQ ID No: 1 are unsubstituted; or
the mutant transaminase has the substitutions Leu61, Ser62, Met65, Phe171, Val266, Lys318, Met320, Pro419, Asn420 and Ser421 and wherein the amino acids at positions corresponding to positions 9, 198, 426 and 464 of SEQ ID No:1 are unsubstituted; or
the mutant transaminase has the substitutions Leu61, Met65, Phe171, Val266, Cys320, Val419, Asn420, Ser421 and Val426 and wherein the amino acids at positions corresponding to positions 9, 62, 198, 318, and 464 of SEQ ID No:1 are unsubstituted; or
the mutant transaminase has the substitutions Tyr9, Leu61, Ser62, Met65, Phe171, Val266, Lys318, Pro419, Asn420 and Ser421 and wherein the amino acids at positions corresponding to positions 198, 320, 426 and 464 of SEQ ID No:1 are unsubstituted; or
the mutant transaminase has the substitutions Tyr9, Leu61, Ser62, Met65, Phe171, Val266, Lys318, Met320, Pro419, Asn420 and Ser421 and wherein the amino acids at positions corresponding to positions 198, 426 and 464 of SEQ ID No:1 are unsubstituted; or
the mutant transaminase has the substitutions Tyr9, Leu61, Met65, Phe171, Val266, Cys320, Val419, Asn420, Ser421 and Val426 and wherein the amino acids at positions corresponding to positions 62, 198, 318, and 464 of SEQ ID No:1 are unsubstituted; or
the mutant transaminase has the substitutions Leu61, Ser62, Met65, Phe171, Asp198, Val266, Lys318, Pro419, Asn420, Ser421 and Met464 and wherein the amino acids at positions corresponding to positions 9, 320, and 426 of SEQ ID No:1 are unsubstituted; or
the mutant transaminase has the substitutions Leu61, Ser62, Met65, Phe171, Asp198, Val266, Lys318, Met320, Pro419, Asn420, Ser421 and Met464 and wherein the amino acids at positions corresponding to positions 9 and 426 of SEQ ID No:1 are unsubstituted; or
the mutant transaminase has the substitutions Leu61, Met65, Phe171, Asp198, Val266, Cys320, Val419, Asn420, Ser421, Val426 and Met464 wherein the amino acids at positions corresponding to positions 9, 62, and 318 of SEQ ID No:1 are unsubstituted.

Still more particularly, the mutant transaminase is characterized as follows:
the mutant transaminase has the substitutions Leu61, Met65, Val 266 and Pro419; or
the mutant transaminase has the substitutions Leu61, Met65, Val266, Val419 and Val426; or
the mutant transaminase has the substitutions Leu61, Ser62, Met65, Phe171, Val 266, Lys318, Pro419, Asn420 and Ser421; or
the mutant transaminase has the substitutions Leu61, Ser62, Met65, Phe171, Val266, Lys318, Met320, Pro419, Asn420 and Ser421; or
I the mutant transaminase has the substitutions Leu61, Met65, Phe171, Val266, Cys320, Val419, Asn420, Ser421 and Val426; or
the mutant transaminase has the substitutions Tyr9, Leu61, Ser62, Met65, Phe171, Val266, Lys318, Pro419, Asn420 and Ser421; or
the mutant transaminase has the substitutions Tyr9, Leu61, Ser62, Met65, Phe171, Val266, Lys318, Met320, Pro419, Asn420 and Ser421; or
the mutant transaminase has the substitutions Tyr9, Leu61, Met65, Phe171, Val266, Cys320, Val419, Asn420, Ser421 and Val426; or
the mutant transaminase has the substitutions Leu61, Ser62, Met65, Phe171, Asp198, Val266, Lys318, Pro419, Asn420, Ser421 and Met464; or
the mutant transaminase has the substitutions Leu61, Ser62, Met65, Phe171, Asp198, Val266, Lys318, Met320, Pro419, Asn420, Ser421 and Met464; or
the mutant transaminase has the substitutions Leu61, Met65, Phe171, Asp198, Val266, Cys320, Val419, Asn420, Ser421, Val426 and Met464.
   wherein the defined substitutions are the only substitutions in the mutant transaminases relative to the amino acid sequence of SEQ ID NO: 1.

In an especially preferred embodiment of the present invention the mutant transaminase of the first aspect of the present invention is as defined above and i) consists of or ii) comprises an amino acid sequence that is at least 85%, 90%, 95%, 96%, 97%, 98%, or 99%, particularly 100% identical to the amino acid sequence of any of SEQ ID NO: 2 to 12.

The term “sequence identity” as used herein describes the percentage of characters that exactly match between two different sequences. For example, the term “at least 85% identical to the amino acid sequence of SEQ ID NO: 2” as used herein means that the amino acid sequence of the mutant transaminase of the present invention has an amino acid sequence characterized in that, within a stretch of 100 amino acids, at least 85 amino acid residues are identical to the sequence of the corresponding sequence of SEQ ID NO: 2. Sequence identity according to the present invention can, e.g., be determined by methods described above.

The mutant transaminase of the first aspect shows a substantially increased reductive amination activity relative to the wild-type transaminase, particularly in the presence of an organic solvent and an amine donor. In the presence of an organic solvent and the amine donor the increase may be at least 2 fold, but may also reach levels of 50-fold or above.

The mutant transaminase of the first aspect may also show increased stability in aqueous organic media, particularly in aqueous media which contain 4.0% v/v to 40.0% v/v of an organic solvent, which builds out a monophasic environment, such as dimethylsulfoxide.

The mutant transaminase of the first aspect may furthermore show increased stability in the presence of an organic solvent and a residual amount of water. It is important for the activity of the mutant transaminase that a residual amount of water, typically between 1.0% wt. and 5.0% wt., particularly between 1.4% wt. to 3.4% wt., more particularly between 1.8% wt. to 2.8% wt. of the total amount of the solvent is present.

Suitable organic solvents are ethers, esters or hydrocarbons. Particular ethers are diethoxymethan or t.butyl methylether. Particular hydrocarbon include toluene and particular esters include lower alkyl esters of acetic acid or propionic acid, such as ethyl acetate, isopropyl acetate, isobutyl acetate, iso-propyl 2-methylpropanoate or tert-butyl 2,2-dimethylpropanoate. Preferred are the named esters with more particular preference for isopropyl acetate.

The mutant transaminase of the first aspect may be capable of reductive amination of a ketone into a primary amine, especially a prochiral ketone into a chiral primary amine. Additionally or alternatively, the mutant transaminase may show reductive amination activity at a temperature of between 40° C. and 70° C., particularly between 50° C. and 70° C.

Ketones suitable applied in the reductive amination can be illustrated by the formula I

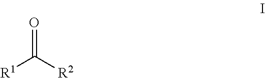

wherein
R¹ and R² independently of each other represent optionally substituted alkyl, aryl, carbocyclyl or heterocyclyl; or
R¹ and R² together with the carbon atom they are attached to form an optionally substituted mono- or poly-cyclic carbocyclic or heterocyclic ring, and
wherein optional substituents are selected from alkyl, alkoxy, aryl, heteroaryl, aryloxy, halogen, hydroxyl or cyano.

In a preferred embodiment, the ketone is a prochiral ketone, wherein R¹ and R² are different.

In a further preferred embodiment
R¹ is optionally substituted C₁₋₁₂-alkyl;
R² is optionally substituted aryl, aryl alkyl, heterocyclyl or heterocyclyl alkyl; or
R¹ and R² together with the carbon atom they are attached to form an optionally substituted mono- or poly-cyclic carbocyclic or heterocyclic ring, and
wherein optional substituents are selected from C₁₋₁₂-alkyl, C₁₋₁₂-alkoxy, aryl, heteroaryl, aryloxy, halogen, hydroxyl or cyano.

The substituents may themselves be further substituted with the substituents outlined above.

Examples of suitable ketones are cylohexanone, 2-hexanone, acetophenone, benzylacetone, 3-acetylpyridine, 3-proprionylpyridine, 5-acetyl-2-chloropyridine, 3-acetyl-5-chloropyridine, 3-acetyl-2-chloropyridine, 5-acetyl-2-methoxy-pyridine, 1-quinolin-3-yl-ethanone, 2-acetlypyrazine, indan-1-one or 1-piperidin-3-yl-ethanone.

A preferred example is the prochiral ketone of formula X. The prochiral ketone of formula X is an intermediate in the synthesis of praisetinib, which is a kinase inhibitor indicated for the treatment of non-small cell lung cancer (NSCLC).

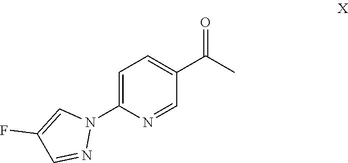

The terms as used herein for the ketone of formula I have the following meaning.

The term “halogen” denotes fluoro, chloro, bromo, or iodo, particularly chlorine or fluorine.

The term “alkyl” denotes a monovalent linear or branched saturated hydrocarbon group of 1 to 12 carbon atoms. In particular embodiments, alkyl has 1 to 7 carbon atoms, and in more particular embodiments 1 to 4 carbon atoms. Examples of alkyl include methyl, ethyl, propyl, iso-propyl, n-butyl, iso-butyl, sec-butyl, or tert-butyl. The most preferred alkyl group for R¹ is methyl.

The term “alkoxy” refers to an alkyl group as defined above which is attached to an oxygen radical. Examples of alkoxy include methoxy, ethoxy, iso-propoxy, n-butoxy, iso-butoxy, sec-butoxy, or tert-butoxy.

The term “aryl” denotes a monovalent aromatic carbocyclic mono- or bicyclic ring system comprising 6 to 10 carbon ring atoms. Examples of aryl moieties include phenyl and naphthyl.

The term “aryl alkyl” denotes aryl substituted alkyl group wherein the definitions for aryl and alkyl are as outlined above.

The term “aryloxy” denotes an aryl group as defined above which is attached to an oxygen radical. A suitable example is phenoxy or naphthyloxy.

The term “mono- or poly-cyclic ring” refers to a compound featuring one or more closed rings of atoms, primarily carbon. These ring substructures include cycloalkanes, aromatics, and other ring types. Though “poly” literally means “many”, it also includes bicyclic, tricyclic, tetracyclic, etc.

The term “carbocyclic” refers to a saturated, partially unsaturated or unsaturated cyclic compound in which all of the ring members are carbon atoms, such as cyclohexane, decaline or 1,2-dihydronaphthalene. The compound may be aromatic or non-aromatic. Simple aromatic rings consist only of a conjugated planar ring system. Typical simple aromatic compounds are benzene, indole, and cyclotetradecaheptaene. Polycyclic aromatic hydrocarbons contain only carbon and hydrogen and are composed of multiple aromatic rings. Examples include naphthalene, anthracene, phenanthrene and indane.

The term “heterocyclyl” refers to a saturated, partially unsaturated or unsaturated 5 to 6 membered monocyclic ring or 8 to 10 membered bicyclic ring, which can comprise 1, 2 or 3 heteroatoms selected from nitrogen, oxygen and/or sulphur. The ring system may be aromatic or non-aromatic. Typical heterocyclyl residues are pyridyl, pyridinyl, pyrazolyl, pyrimidinyl, benzoimidazolyl, quinolinyl and isoquinolinyl, thienyl, furyl, pyrrolyl, pyrazolyl, isoxazolyl, oxazolyl, thiazolyl or imidazolyl.

The term “heterocyclyl alkyl” denotes a heterocyclyl substituted alkyl group wherein the definitions for heterocyclyl and alkyl are as outlined above.

Optional substituents as used herein for the ketone of formula I can be selected from alkyl, alkoxy, aryl, aryloxy, halogen, hydroxyl or cyano. The preferences and examples as outlined above apply for the substituents as well.

In a second aspect, the present invention relates to a nucleic acid coding for the mutant transaminase of the first aspect, optionally comprised in a vector.

The term “nucleic acid” as used herein generally relates to any nucleotide molecule which encodes the mutant transaminase of the invention and which may be of variable length. Examples of a nucleic acid of the invention include, but are not limited to, plasmids, vectors, or any kind of DNA and/or RNA fragment(s) which can be isolated by standard molecular biology procedures, including, e.g. ion-exchange chromatography. A nucleic acid of the invention may be used for transfection or transduction of a particular cell or organism.

Nucleic acid molecule of the present invention may be in the form of RNA, such as mRNA or cRNA, or in the form of DNA, including, for instance, cDNA and genomic DNA e.g. obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand. Nucleic acid molecule as used herein also refers to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA.

Additionally, the nucleic acid may contain one or more unusual bases, such as inosine, or modified bases, such as tritylated bases, to name just two examples. Such nucleic acids may also contain modifications e.g. in the ribose-phosphate backbone to increase stability and half life of such molecules in physiological environments. Thus, DNAs or RNAs with backbones modified for stability or for other reasons are “nucleic acid molecule” as that feature is intended herein. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia.

Furthermore, the nucleic acid molecule encoding the mutant transaminase of the invention can be functionally linked, using standard techniques such as standard cloning techniques, to any desired sequence, such as a regulatory sequence, leader sequence, heterologous marker sequence or a heterologous coding sequence to create a fusion protein.

The nucleic acid of the invention may be comprised in an expression vector, wherein the nucleic acid is operably linked to a promoter sequence capable of promoting the expression of the nucleic acid in a host cell

In a third aspect, the present invention relates to a cell comprising the mutant transaminase of the first aspect of the present invention and/or the nucleic acid of the second aspect of the present invention. The cell may be referred to as host cell and comprise the transaminase of the first aspect of the present invention, optionally as part of a fusion protein linked to another molecule, or the nucleic acid of the second aspect of the present invention, optionally comprised in an expression vector. The cultivation of host cells according to the invention is a routine procedure known to the skilled person. That is, a nucleic acid encoding a mutant transaminase of the invention can be introduced into a suitable host cell(s) to produce the respective protein by recombinant means. These host cells can by any kind of suitable cells, preferably bacterial cells such as E. coli, which can be cultivated in culture. After the protein has been expressed in the respective host cell, the cells can be harvested and serve as the starting material for the preparation of a cell extract containing the protein of interest. A cell extract containing the protein of interest may be obtained by lysis of the cells. Methods of preparing a cell extract by means of either chemical or mechanical cell lysis are well known to the person skilled in the art, and include, but are not limited to, e.g. hypotonic salt treatment, homogenization, or ultrasonification. In an preferred embodiment of the present invention, the host cell, particularly E. coli cells comprising the mutant transaminase according to the first aspect, are spray-dried and used in the methods of the present invention described in the following.

In a further aspect the present invention relates to a method for the enzymatic reductive transamination of a ketone and the formation of a primary amine in the presence of mutant transaminase of the present invention, as described above. Preferably, the ketone is a prochiral ketone and the primary amine is a chiral primary amine.

As hereinbefore described suitable ketones have the formula I

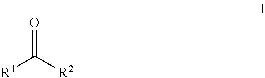

wherein
R¹ and R² independently of each other represent optionally substituted alkyl, aryl, carbocyclyl or heterocyclyl; or
R¹ and R² together with the carbon atom they are attached to form an optionally substituted mono- or poly-cyclic carbocyclic or heterocyclic ring, and
wherein optional substituents are selected from alkyl, alkoxy, aryl, heteroaryl, aryloxy, halogen, hydroxyl or cyano
   and the resulting primary amine has the formula II

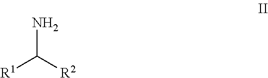

wherein
R¹ and R² independently of each other represent optionally substituted alkyl, aryl, carbocyclyl or heterocyclyl; or
R¹ and R² together with the carbon atom they are attached to form an optionally substituted mono- or poly-cyclic carbocyclic or heterocyclic ring, and
wherein optional substituents are selected from alkyl, alkoxy, aryl, heteroaryl, aryloxy, halogen, hydroxyl or cyano.

The preferred embodiments as described above also apply for the primary amine of formula II.

The enzymatic reductive transamination of the preferred prochiral ketone of formula X results in the chiral primary amine of formula XI.

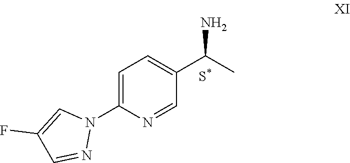

The enzymatic reductive amination typically takes place in the presence of an organic solvent.

It is however important for the activity of the enzyme that a residual amount of water, typically between 1.0% wt. and 5.0% wt., particularly between 1.4% wt. to 3.4% wt., more particularly between 1.8% wt. to 2.8% wt. of the total amount of the solvent is present.

The organic solvent together with the residual water preferably builds a monophasic system, which is also known as a micro aqueous reaction system (MARS).

Therefore, organic solvents are selected according to their capability to build out such a monophasic environment.

Suitable organic solvents are ethers, esters or hydrocarbons. Particular ethers are diethoxymethan or t.butyl methylether. Particular hydrocarbon include toluene and particular esters include lower alkyl esters of acetic acid or propionic acid, such as ethyl acetate, isopropyl acetate, isobutyl acetate, iso-propyl 2-methylpropanoate or tert-butyl 2,2-dimethylpropanoate. Preferred organic solvents are the named esters with more particular preference for isopropyl acetate.

Alternatively, but less preferred, the enzymatic reductive amination can be performed in aqueous organic media, particularly in aqueous media which contain 4.0% v/v to 40.0% v/v of an organic solvent which build out a monophasic environment, such as dimethylsulfoxide.

The transamination reaction requires a suitable amine donor, which upon its transformation in the respective ketone can easily be separated from the desired primary amine.

The amine donor may be a primary aliphatic amine. As a rule primary aliphatic amines, such as isopropyl amine or phenylethyl amine are applied. Preferred amine donor is isopropyl amine.

The amine donor is as a rule used in amounts of 2 eq. to 20 eq., preferably 4 eq. to 8 eq., more preferably 4 eq. to 6 eq.

The transaminase mutant enzyme is typically provided in dry formulations, which enables defined water content and ensures high activity in the micro aqueous reaction system (MARS).

The transaminase mutant enzyme can be applied as lyophilized or spray-dried enzyme powder, respectively as enzyme immobilisate or as whole cells, such as E. coli cells, containing the overexpressed transaminase. Whole E. coli cells are preferred due to their higher stability.

The substrate to enzyme ratio (s/e weight ratio) is usually between 1 and 5, preferably between 2 and 3. For E. coli cells powder the s/e weight ratio usually is between 1 and 5, preferably between 3 and 4.

The reaction temperature depends on the stability of the transaminase mutant enzyme formulation and the reaction environment. As a rule, the reaction temperature is chosen between 40° C. and 70° C.

For enzyme powder the reaction temperature can be chosen between 40° C. and 65° C., preferably between 45° C. and 60° C., more preferably at about 50° C. or 55° C. For whole E. coli cell powder the reaction temperature is selected between 40° C. and 70° C., preferably between 55° C. and 65° C., more preferably at about 60° C. or 65° C.

The ketone substrate loading in % wt. can be selected between 1% and 15%, preferably between 2% and 10%, more preferably at about 5%.

The reaction equilibrium can be further shifted towards product (primary amine) formation by in-situ product removal and/or by continuous removal of the ketone byproduct (formed from the amine donor).

For example the primary amine of formula II can be precipitated as an insoluble salt, e.g. as the hydrochloride salt and/or the ketone byproduct can be distilled of.

The isolation of the resulting primary amine can be accomplished following methods well known to the skilled in the art.

Unless defined otherwise, all technical and scientific terms and any acronyms used herein have the same meanings as commonly understood by one of ordinary skill in the art in the field of the invention. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The invention is not limited to the particular methodology, protocols, and reagents described herein because they may vary. Although any methods and materials similar or equivalent to those described herein can be used in the practice of the present invention, the preferred methods, and materials are described herein. Further, the terminology used herein is for the purpose of describing particular embodiments only and is not intended to limit the scope of the present invention.

As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively. Similarly, the word “or” is intended to include “and” unless the context clearly indicates otherwise. The term “plurality” refers to two or more.

The following Examples are intended to illustrate various embodiments of the invention. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### EXAMPLE 1: ENZYME PRODUCTION

### Cultivation, Protein Expression, Cell Harvesting or Cell Lysis

Cultivation in automation platform: Bacterial cells were cultivated in 96-well microtiter plates (ThermoFisher Scientific) using a Tecan Fluent automation platform. Precultures were grown from fresh single transformants in 160 μL LB medium supplemented with 50 mg/L Kanamycin and incubated at 30° C., 850 rpm for 20 h. Ten microliters (10 μL) of the overnight preculture were inoculated into 180 μL ZYM-5052 autoinduction medium without trace elements (10 g/L peptone, 5 g/L yeast extract, 5 g/L glycerol, 0.55 g/L glucose monohydrate, 2.1 g/L lactose monohydrate, 10.6 g/L sodium phosphate dibasic salt, 3.4 g/L potassium phosphate monobasic salt, 2.15 g/L ammonium chloride, 0.59 g/L sodium chloride, 0.663 g/L ammonium sulfate, 2 mM magnesium sulfate) supplemented with 50 mg/L kanamycin. The cultures were grown at 20° C., 800 rpm for 22 h. Automated cell harvesting proceeded by plate centrifugation at 4° C., 4060 rpm for 30 min, followed by supernatant removal by manual inversion of the plates. Cell pellets were stored at −20° C. for their use in reductive amination reactions.

Manual cultivation in 96-deepwell plates: Bacterial cells were cultivated in a 96-deepwell plate (DWP) format with V-bottom shape and conical base aids. All plates contained strains harboring the negative control plasmid as well as the parental enzymes or positive controls. Precultures were started by inoculation of fresh single transformants into LB medium containing 50 mg/L kanamycin, followed by incubation at 30° C. with shaking at 300 rpm for 18 h (Duetz system, Kühner shaker). Main cultures in DWP were started by inoculation of 8 μL pre-culture into 500 μl ZYM-5052 autoinduction medium without trace elements containing 50 mg/L kanamycin. The cultures were incubated at 20° C., 300 rpm for 20 h without humidity (Duetz system, Kühner shaker). Cells were pelleted by centrifugation at 4° C., 3,300 g for 20 min.

Biocatalyst preparation for reactions in aqueous media: Cell pellets after one freeze-thaw cycle were used for reductive amination reactions. Alternatively, cells were lysed by adding 0.2 ml of 10 mM HEPES buffer pH 7 containing 1 mg/mL lysozyme from chicken egg white, 0.75 mg/mL polymyxin B and 0.2 mg/mL DNase I. Cell suspensions were incubated at 30° C. with shaking at 300 rpm for 1 h (Duetz system, Kühner shaker), followed by centrifugation at 4° C., 3,300 g for 30 min. Freshly extracted supernatants were used for reductive amination reactions in aqueous buffer.

### EXAMPLE 2: SCREENING OF ENZYME ACTIVITY AND SELECTIVITY

Micro-scale reactions in aqueous media (4-40% DMSO): Biocatalytic reactions in aqueous media were carried out in a total reaction volume of 0.2 mL in 2 mL Eppendorf tubes or 96-DWP. Samples contained 0.2% or 1% [w/v] substrate, 4-40% [v/v] DMSO, 0.5-2 M isopropylamine hydrochloride, 2 mM pyridoxal 5′-phosphate monohydrate and 0.2 M HEPES buffer pH 7. Biomass or lysate concentrations were adjusted along the evolution campaign to accurately compare the investigated evolved enzyme variant against its parental enzyme. Unless otherwise specified, samples were incubated at 50° C. with shaking at 1000 rpm (Eppendorf thermomixer) or at 300 rpm (Kühner Kelvin+shaker) in a fume hood. Reactions were quenched with acetonitrile in a sample-solvent ratio of 1:2 or 1:5 (final acetonitrile concentration of 50-62.5% in water), mixed for 5-10 min on a thermomixer or by pipetting, and centrifuged at RT, 13,500 g for 2 min (tubes) or at 10° C., 3,330 g for 10 min (DWP). Supernatants were transferred from tubes to glass vials or measured in plate format using either a chiral or an accelerated achiral HPLC method.

Micro-scale reactions in organic media: Small-scale reactions were carried out in 2 mL Eppendorf tubes in a final volume of 0.5 or 1 mL. Samples contained 1% or 2% [w/v] substrate, 5-9 eq isopropylamine free base, and water-saturated isopropyl acetate. The organic media was initially prepared by mixing 10 mL isopropyl acetate with 0.3 mL water containing 1 M potassium phosphate buffer pH 7, 5 M isopropylamine hydrochloride and 2 m pyridoxal 5′-phosphate monohydrate for 1 h. At a later stage, isopropyl acetate was saturated only with water, as the reactions were observed to proceed without buffer or added pyridoxal 5′-phosphate monohydrate. Lyophilized cells or lyophilized enzyme lysates were used at a ratio suitable for each evolution round. Samples were incubated at 50-70° C. with shaking. After a determined reaction time, 100 μL of the reaction mix was diluted with 900 μL dimethylacetamide and centrifuged. Supernatants were analyzed using the corresponding HPLC-UV method.

Substrate and Product: The ketone substrate 1-(6-(4-fluoro-1H-pyrazol-1-yl)pyridin-3-yl) ethan-1-one of the formula X has been employed

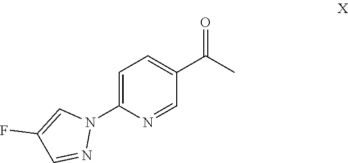

and the resulting chiral primary amine is(S)-1-(6-(4-fluoro-1H-pyrazol-1-yl)pyridin-3-yl) ethan-1-amine of formula XI.

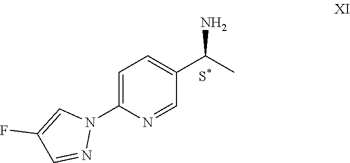

Chiral HPLC analysis of aqueous reactions (4-40% DMSO): Samples from reactions in aqueous media were measured by HPLC-UV at 269 nm on an Agilent 1290 HPLC instrument coupled to DAD and equipped with a chiral Daicel CrownPak CR-I (+) column (3 mm, 150 mm, 5 μm). The flow and column temperature were adjusted to 0.8 mL/min and 30° C. The injection volume was 1 μL. Mobile phase A consisted of 95% [v/v] Millipore water+5% [v/v] acetonitrile+0.25% [v/v] trifluoroacetic acid (TFA), while mobile phase B consisted of acetonitrile+0.25% [v/v] TFA. The method was isocratic: 0-4.5 min, 30% B. Conversions were calculated from product formation, using a calibration curve of XI and the initial concentration of X. Fold-increase over wild type (FIOWT) or over the parent (FIOP) values were calculated as Peak area of XI (variant)/Peak area of XI (wild-type or parent), accordingly.

Chiral HPLC analysis of organic reactions: Samples from reactions in organic media were measured using an HPLC-UV system and a column with the same characteristics described above, but with the following method modifications: The flow and column temperature were adjusted to 1 mL/min and 25° C. The injection volume was 1-5 μL. Mobile phase A consisted of 0.31% (v/v) perchloric acid in Millipore water, while mobile phase B consisted of acetonitrile. The method was isocratic: 0-10 min, 20% B. Conversions were calculated from the relative areas of X and XI.

Accelerated achiral UHPLC-UV analysis of aqueous reactions (4-40% DMSO): Samples from reactions in aqueous media were measured on an Agilent 1290 HPLC-DAD/MS instrument equipped with an InfinityLab Poroshell 120 EC-C18 Guard column (2.1 mm, 5 mm, 1.9 μm). The guard column was assembled as a regular analytical column for DAD analysis without MS detection. The flow and column temperature were adjusted to 1 mL/min and 30° C. The injection volume was 1 μL. Mobile phase A consisted of 95% [v/v] Millipore water+5% [v/v] acetonitrile+0.25% [v/v] TFA, while mobile phase B consisted of acetonitrile+0.25% [v/v] TFA. The following gradient was applied: 0-0.1 min, 0% B; 0.1-0.25 min, 100% B; 0.25-0.34 min, 100% B, 0.34-0.35; 0% B. The enantioselectivity of the engineered enzyme hits was always verified using the chiral HPLC-UV method previously described and reported as enantiomeric excess (ee) for product X. Conversion, product titers and FIOWT/FIOP values were calculated as previously described as well.

### Results

In seven rounds of transaminase optimization, a set of promising mutation sides had been identified. For further analysis, the top variants, mainly based on their performance after 20 h, were selected. The following 12 enzyme variants as well as the wild-type transaminase of SEQ ID NO: 1 further investigated:
M61L_W65M_I266V_R419V
W65G_R419V_I426V
M61L_W65M_I266V_R419P
M61L_W65G_R419V_I426V
M61L_W65F_I266V_D349Y_R419P_I426V
M61L_W65M_I266V_R419V_I426V
W65F_R419P_I426V
W65C_R419P_I426V
W65F_R419P
W65M_I266V_R419P_I426V
W65M_R419V_I426V
M61L_W65F_I266V_R419P_I426V_K463T

In the above mutants, the numbers indicate the amino acid position in SEQ ID NO:1. The letter prior to the number specifies the amino acid of the wild-type transaminase of SEQ ID NO: 1, and the letter following the number indicates the amino acid, which has been used in the substitution of the amino acid of the wild-type.

The two best enzymes were selected as parental enzymes. Among the R419V-containing variants, M61L_W65M_I266V_R419V_I426V (LMVVV, SEQ ID NO:3) yielded the highest conversion levels at 30% DMSO after 20 h (relative to biomass concentration) It was also the fastest and one of the most tolerant towards 40% DMSO. Among the R419P-containing variants, M61L_W65M_I266V_R419P (LMVPI, SEQ ID NO:2) yielded the highest conversion levels at 30% DMSO after 20 h (relative to biomass concentration).

In the following, the results of experiments with various transaminase mutants having single or multiple substitutions are shown:

TABLE 1
Variants with single mutation at position
9 (Single site mutagenesis)

| AA | FIOWT |
|---|---|
| H—His (wild-type) | 1.0 |
| Y—Tyr | 6.8 |

Aqueous reactions contained 1% [w/v] X, one-time freeze-thawed cells (robot cultivated cells resuspended in 50 μL buffer, and 40 μL thereof used), 2M isopropylamine hydrochloride, 30% [v/v] DMSO, and were incubated at 50° C. for 20 h. ee (XI) >99.5%.

TABLE 2
Variants with single mutation at position 9 and at
least one further mutation
(Combinatorial site mutagenesis)

| His | Met | Ala | Trp | Met | lle | Glu | Asn |
|---|---|---|---|---|---|---|---|
| 9 | 61 | 62 | 65 | 171 | 266 | 318 | 320 |
| H-His | M-Met | A-Ala | W-Trp | M-Met | I-Ile | E-Glu | N-Asn |
| H-His | L-Leu | A-Ala | M-Met | M-Met | V-Val | E-Glu | N-Asn |
| Y-Tyr | L-Leu | A-Ala | M-Met | M-Met | V-Val | E-Glu | N-Asn |
| H-His | L-Leu | S-Ser | M-Met | F-Phe | V-Val | K-Lys | N-Asn |
| Y-Tyr | L-Leu | S-Ser | M-Met | F-Phe | V-Val | K-Lys | N-Asn |
| H-His | L-Leu | S-Ser | M-Met | F-Phe | V-Val | K-Lys | M-Met |
| Y-Tyr | L-Leu | S-Ser | M-Met | F-Phe | V-Val | K-Lys | M-Met |
| H-His | L-Leu | A-Ala | M-Met | F-Phe | V-Val | E-Glu | C-Cys |
| Y-Tyr | L-Leu | A-Ala | M-Met | F-Phe | V-Val | E-Glu | C-Cys |
| | | | Arg | His | Val | Ile | |
| | | | 419 | 420 | 421 | 426 | FIOWT |
| | | | R-Arg | H-His | V-Val | I-Ile | 1 |
| | | | V-Val | H-His | V-Val | V-Val | 281 |
| | | | V-Val | H-His | V-Val | V-Val | 348 |
| | | | V-Val | N-Asn | S-Ser | I-Ile | 428 |
| | | | V-Val | N-Asn | S-Ser | I-Ile | 524 |
| | | | V-Val | N-Asn | S-Ser | I-Ile | 425 |
| | | | V-Val | N-Asn | S-Ser | I-Ile | 535 |
| | | | V-Val | N-Asn | S-Ser | V-Val | 419 |
| | | | V-Val | N-Asn | S-Ser | V-Val | 506 |

Aqueous reactions contained 1% [w/v] X, one-time freeze-thawed cells (manually cultivated cells in 0.5 mL, OD600 ~ 10, 17 μL thereof used), 2M isopropylamine hydrochloride, 30% [v/v] DMSO, and were incubated at 50°C. for 20 h. In all cases, ee (XI) > 99.5%.

TABLE 3
Variants with single mutation at position 62 and at
least one further mutation
(Combinatorial site mutagenesis)

| Met | Ala | Trp | Met | Ile | Glu | Asn |
|---|---|---|---|---|---|---|
| 61 | 62 | 65 | 171 | 266 | 318 | 320 |
| L-Leu | A-Ala | M-Met | M-Met | V-Val | E-Glu | N-Asn |
| L-Leu | S-Ser | M-Met | F-Phe | V-Val | E-Glu | L-Leu |
| L-Leu | S-Ser | M-Met | F-Phe | V-Val | E-Glu | M-Met |
| L-Leu | S-Ser | M-Met | M-Met | V-Val | E-Glu | L-Leu |
| L-Leu | S-Ser | M-Met | F-Phe | V-Val | E-Glu | K-Lys |
| L-Leu | S-Ser | M-Met | F-Phe | V-Val | E-Glu | C-Cys |
| L-Leu | S-Ser | M-Met | F-Phe | V-Val | K-Lys | M-Met |
| L-Leu | S-Ser | M-Met | F-Phe | V-Val | K-Lys | N-Asn |
| L-Leu | S-Ser | M-Met | F-Phe | V-Val | K-Lys | N-Asn |
| L-Leu | A-Ala | M-Met | M-Met | V-Val | E-Glu | N-Asn |
| L-Leu | S-Ser | M-Met | M-Met | V-Val | K-Lys | N-Asn |
| L-Leu | S-Ser | M-Met | F-Phe | V-Val | E-Glu | L-Leu |
| L-Leu | S-Ser | M-Met | M-Met | V-Val | K-Lys | M-Met |
| L-Leu | S-Ser | M-Met | F-Phe | V-Val | K-Lys | C-Cys |
| L-Leu | S-Ser | M-Met | F-Phe | V-Val | E-Glu | N-Asn |
| L-Leu | S-Ser | M-Met | W-Trp | V-Val | K-Lys | N-Asn |
| L-Leu | S-Ser | M-Met | M-Met | V-Val | E-Glu | M-Met |
| L-Leu | S-Ser | M-Met | F-Phe | V-Val | E-Glu | M-Met |
| L-Leu | S-Ser | M -Met | M-Met | V-Val | E-Glu | M-Met |
| L-Leu | S-Ser | M-Met | M-Met | V-Val | K-Lys | L-Leu |
| L-Leu | S-Ser | M-Met | F-Phe | V-Val | E-Glu | L-Leu |
| L-Leu | S-Ser | M-Met | M-Met | V-Val | K-Lys | C-Cys |
| L-Leu | S-Ser | M-Met | M-Met | V-Val | K-Lys | N-Asn |
| L-Leu | S-Ser | M-Met | F-Phe | V-Val | E-Glu | L-Leu |
| L-Leu | S-Ser | M-Met | M-Met | V-Val | K-Lys | N-Asn |
| L-Leu | S-Ser | M-Met | M-Met | V-Val | K-Lys | M-Met |
| | | Arg | His | Val | Ile | Conv. |
| | | 419 | 420 | 421 | 426 | [%] |
| | | P-Pro | H-His | V-Val | I-Ile | 5.4 |
| | | P-Pro | H-His | V-Val | I-Ile | 5.9 |
| | | P-Pro | N-Asn | S-Ser | I-Ile | 6.1 |
| | | P-Pro | S-Ser | S-Ser | I-Ile | 8.4 |
| | | P-Pro | N-Asn | S-Ser | I-Ile | 9.0 |
| | | P-Pro | S-Ser | S-Ser | I-Ile | 9.5 |
| | | P-Pro | N-Asn | S-Ser | I-Ile | 11.7 |
| | | P-Pro | N-Asn | S-Ser | I-Ile | 13.0 |
| | | P-Pro | A-Ala | S-Ser | I-Ile | 13.5 |
| | | V-Val | H-His | V-Val | V-Val | 3.2 |
| | | V-Val | N-Asn | S -Ser | V-Val | 5.5 |
| | | V-Val | N-Asn | S-Ser | V-Val | 6.1 |
| | | V-Val | D-Asp | S-Ser | V-Val | 6.3 |
| | | V-Val | N-Asn | S-Ser | V-Val | 6.6 |
| | | V-Val | D-Asp | S-Ser | V-Val | 6.7 |
| | | V-Val | N-Asn | S-Ser | V-Val | 6.9 |
| | | V-Val | S-Ser | S-Ser | V-Val | 7.0 |
| | | V-Val | S-Ser | S-Ser | V-Val | 7.9 |
| | | V-Val | N-Asn | S-Ser | V-Val | 8.0 |
| | | V-Val | H-His | S-Ser | V-Val | 8.0 |
| | | V-Val | A-Ala | S-Ser | V-Val | 8.1 |
| | | V-Val | H-His | S-Ser | V-Val | 8.3 |
| | | V-Val | N-Asn | S-Ser | V-Val | 8.9 |
| | | V-Val | H-His | S-Ser | V-Val | 9.2 |
| | | V-Val | N-Asn | S-Ser | V-Val | 10.2 |
| | | V-Val | N-Asn | S-Ser | V-Val | 12.3 |

Organic reactions contained 1% [w/v] X, lyophilized cells (~17 mg cells wet weight (CWW) from 0.5 mL cultures in deep-well plates), water-saturated isopropyl acetate, 5 eq isopropylamine free base, and were incubated at 60º C. for 4 h. In all cases, ee (XI) > 99.5%.

TABLE 4
Variants with single mutation at position
61 (Single site mutagenesis)

| AA | FIOWT |
|---|---|
| M—Met (wild-type) | 1.00 |
| L—Leu | 2.54 |

Aqueous reactions contained 0.2% [w/v] X, 34% [v/v] lysate, 1M isopropylamine hydrochloride, 4% [v/v] DMSO, and were incubated at 50° C. for 2 h. ee (XI) >99.5%.

TABLE 5
Variants with single mutation at position 61 and at least
one further mutation (Combinatorial site mutagenesis)

| Met 61 | Trp 65 | Ile 266 | Arg 419 | Ile 426 | FIOWT |
|---|---|---|---|---|---|
| M—Met | W—Trp | I—Ile | R—Arg | I—Ile | 1 |
| L—Leu | M—Met | V—Val | V—Val | V—Val | 104 |
| L—Leu | G—Gly | I—Ile | V—Val | V—Val | 109 |
| L—Leu | M—Met | V—Val | P—Pro | I—Ile | 109 |
| L—Leu | M—Met | V—Val | V—Val | I—Ile | 114 |

Aqueous reactions contained 1% [w/v] X, one-time freeze-thawed cells (manually cultivated cells in 0.5 mL, OD600 ~10, resuspended in 200 μL buffer, 50 μL thereof used), 2M isopropylamine hydrochloride, 30% [v/v] DMSO, and were incubated at 50° C. for 20 h. In all cases, ee (XI) >99.5%.

TABLE 6
Variants with single mutation at position
65 (Single site mutagenesis)

| AA | FIOWT |
|---|---|
| W—Trp (wild-type) | 1.00 |
| G—Gly | 1.35 |
| S—Ser | 1.40 |
| A—Ala | 1.46 |
| M—Met | 3.10 |
| L—Leu | 3.39 |
| F—Phe | 3.54 |
| V—Val | 3.58 |
| C—Cys | 3.60 |

Aqueous reactions contained 0.2% [w/v] X, 34% [v/v] lysate, 1M isopropylamine hydrochloride, 4% [v/v] DMSO, and were incubated at 50° C. for 2 h. In all cases, ee (XI) >99.5%.

TABLE 7
Variants with single mutation at position 65 and at least
one further mutation (Combinatorial site mutagenesis)

| Met 61 | Trp 65 | Ile 266 | Arg 419 | Ile 426 | FIOWT |
|---|---|---|---|---|---|
| M—Met | W—Trp | I—Ile | R—Arg | I—Ile | 1 |
| M—Met | A—Ala | I—Ile | V—Val | V—Val | 41 |
| L—Leu | L—Leu | C—Cys | V—Val | V—Val | 54 |
| M—Met | V—Val | C—Cys | A—Ala | V—Val | 55 |
| L—Leu | F—Phe | C—Cys | V—Val | V—Val | 58 |
| M—Met | S—Ser | C—Cys | C—Cys | V—Val | 61 |
| M—Met | S—Ser | V—Val | P—Pro | V—Val | 67 |
| M—Met | F—Phe | I—Ile | P—Pro | I—Ile | 68 |
| M—Met | M—Met | V—Val | C—Cys | I—Ile | 71 |
| M—Met | M—Met | I—Ile | V—Val | V—Val | 72 |
| L—Leu | G—Gly | C—Cys | P—Pro | V—Val | 79 |
| M—Met | M—Met | V—Val | P—Pro | V—Val | 81 |
| L—Leu | C—Cys | I—Ile | V—Val | V—Val | 88 |
| M—Met | C—Cys | I—Ile | P—Pro | V—Val | 94 |
| M—Met | M—Met | I—Ile | P—Pro | L—Leu | 94 |
| M—Met | F—Phe | C—Cys | P—Pro | I—Ile | 96 |
| M—Met | G—Gly | I—Ile | V—Val | V—Val | 111 |

Aqueous reactions contained 1% [w/v] X, one-time freeze-thawed cells (manually cultivated cells in 0.5 mL, OD600 ~10, resuspended in 200 μL buffer, 50 μL thereof used), 2M isopropylamine hydrochloride, 30% [v/v] DMSO, and were incubated at 50° C. for 20 h. In all cases, ee (XI) >99.5%.

TABLE 8
Variants with single mutation at position
171 (Single site mutagenesis)

| AA | FIOWT |
|---|---|
| M—Met (wild-type) | 1.0 |
| F—Phe | 2.1 |
| W—Trp | 2.3 |

Aqueous reactions contained 1% [w/v] X, one-time freeze-thawed cells (robot cultivated cells resuspended in 50 μL buffer, and 40 μL thereof used), 2M isopropylamine hydrochloride, 30% [v/v] DMSO, and were incubated at 50° C. for 20 h. In all cases, ee (XI) > 99.5%.

TABLE 9
Variants with single mutation at position
171 and at least one further mutation
(Combinatorial site mutagenesis)

| Met | Ala | Trp | Met | Ile | Glu | Asn |
|---|---|---|---|---|---|---|
| 61 | 62 | 65 | 171 | 266 | 318 | 320 |
| L-Leu | A-Ala | M-Met | M-Met | V-Val | E-Glu | N-Asn |
| L-Leu | A-Ala | M-Met | F-Phe | V-Val | K-Lys | M-Met |
| L-Leu | S-Ser | M-Met | F-Phe | V-Val | K-Lys | N-Asn |
| L-Leu | S-Ser | M-Met | F-Phe | V-Val | K-Lys | M-Met |
| L-Leu | S-Ser | M-Met | F-Phe | V-Val | K-Lys | N-Asn |
| L-Leu | A-Ala | M-Met | M-Met | V-Val | E-Glu | N-Asn |
| L-Leu | A-Ala | M-Met | W-Trp | V-Val | K-Lys | L-Leu |
| L-Leu | A-Ala | M-Met | F-Phe | V-Val | K-Lys | M-Met |
| L-Leu | A-Ala | M-Met | F-Phe | V-Val | E -Glu | C-Cys |
| | | Arg | His | Val | Ile | Conv. |
| | | 419 | 420 | 421 | 426 | [%] |
| | | P-Pro | H-His | V-Val | I-Ile | 18.3 |
| | | P-Pro | C-Cys | S-Ser | I-Ile | 29.8 |
| | | P-Pro | A-Ala | S-Ser | I-Ile | 50.5 |
| | | P-Pro | N-Asn | S-Ser | I-Ile | 54.2 |
| | | P-Pro | N-Asn | S-Ser | I-Ile | 55.3 |
| | | V-Val | H-His | V-Val | V-Val | 10.6 |
| | | V-Val | S-Ser | S-Ser | V-Val | 27.0 |
| | | V-Val | H-His | S-Ser | V-Val | 33.3 |
| | | V-Val | N-Asn | S-Ser | V-Val | 57.1 |

Organic reactions contained 1% [w/v] X, lyophilized cells (~17 mg CWW from 0.5 mL cultures in deep-well plates), water-saturated isopropyl acetate, 5 eq isopropylamine free base, and were incubated at 60° C. for 20 h. In all cases, ee (XI) > 99.5%.

TABLE 10
Variants with single mutation at position
198 (Single site mutagenesis)

| AA | FIOWT |
|---|---|
| D—Asp (wild-type) | 1.0 |
| L—Leu | 7.4 |
| M—Met | 6.4 |

Aqueous reactions contained 1% [w/v] X, one-time freeze-thawed cells (robot cultivated cells resuspended in 50 μL buffer, and 40 μL thereof used), 2M isopropylamine hydrochloride, 30% [v/v] DMSO and were incubated at 50° C. for 20 h. In all cases, ee (XI) > 99.5%.

TABLE 11
Variants with single mutation at position
198 and at least one further mutation
(Combinatorial site mutagenesis)

| Met | Ala | Trp | Met | Asp | lle | Glu | Asn |
|---|---|---|---|---|---|---|---|
| 61 | 62 | 65 | 171 | 198 | 266 | 318 | 320 |
| M-Met | A-Ala | W-Trp | M-Met | D-Asp | I-Ile | E-Glu | N-Asn |
| L-Leu | A-Ala | M-Met | M-Met | D-Asp | V-Val | E-Glu | N-Asn |
| L-Leu | A-Ala | M-Met | M-Met | L-Asp | V-Val | E-Glu | N-Asn |
| L-Leu | S-Ser | M -Met | F-Phe | D-Asp | V-Val | K-Lys | N-Asn |
| L-Leu | S-Ser | M-Met | F-Phe | L-Leu | V-Val | K-Lys | N-Asn |
| L-Leu | S-Ser | M-Met | F-Phe | D-Asp | V-Val | K-Lys | M-Met |
| L-Leu | S-Ser | M-Met | F-Phe | L-Leu | V-Val | K-Lys | M-Met |
| L-Leu | A-Ala | M-Met | F-Phe | D-Asp | V-Val | E-Glu | C-Cys |
| L-Leu | A-Ala | M-Met | F-Phe | L-Leu | V-Val | E-Glu | C-Cys |
| | | Arg | His | Val | Ile | Ser | |
| | | 419 | 420 | 421 | 426 | 464 | FIOWT |
| | | R-Arg | H-His | V-Val | I-Ile | S-Ser | 1 |
| | | V-Val | H-His | V-Val | V-Val | S-Ser | 281 |
| | | V-Val | H-His | V-Val | V-Val | M-Met | 299 |
| | | V-Val | N-Asn | S-Ser | I-Ile | S-Ser | 428 |
| | | V-Val | N-Asn | S-Ser | I-Ile | M-Met | 437 |
| | | V-Val | N-Asn | S-Ser | I-Ile | S-Ser | 425 |
| | | V-Val | N-Asn | S-Ser | I-Ile | M-Met | 434 |
| | | V-Val | N-Asn | S-Ser | V-Val | S-Ser | 419 |
| | | V-Val | N-Asn | S-Ser | V-Val | M-Met | 419 |

Aqueous reactions contained 1% [w/v] X, one-time freeze-thawed cells (manually cultivated cells in 0.5 mL, OD600 ~10, 17 μL thereof used), 2M isopropylamine hydrochloride, 30% [v/v] DMSO, and were incubated at 50° C. for 20 h. In all cases, ee (XI) > 99.5%.

TABLE 12
Variants with single mutation at position
266 (Single site mutagenesis)

| AA | FIOWT |
|---|---|
| I—Ile (wild-type) | 1.00 |
| V—Val | 1.23 |
| C—Cys | 1.49 |

Aqueous reactions contained 0.2% [w/v] X, 34% [v/v] lysate, 1M isopropylamine hydrochloride, 4% [v/v] DMSO and were incubated at 50° C. for 2 h. In all cases, ee (XI) > 99.5%.

TABLE 13
Variants with mutation at position 266 and at least
one further mutation (Combinatorial site mutagenesis)

| Met 61 | Trp 65 | Ile 266 | Arg 419 | Ile 426 | FIOWT |
|---|---|---|---|---|---|
| M—Met | W—Trp | I—Ile | R—Arg | I—Ile | 1 |
| L—Leu | M—Met | C—Cys | V—Val | V—Val | 63 |
| M—Met | L—Leu | V—Val | P—Pro | V—Val | 69 |
| M—Met | C—Cys | V—Val | P—Pro | I—Ile | 78 |
| M—Met | C—Cys | C—Cys | P—Pro | V—Val | 87 |

Aqueous reactions contained 1% [w/v] X, one-time freeze-thawed cells (manually cultivated cells in 0.5 mL, OD600~10, resuspended in 200 μL buffer, 50 μL thereof used), 2M isopropylamine hydrochloride, 30% [v/v] DMSO, and were incubated at 50° C. for 20 h. In all cases, ee (XI) > 99.5%.

TABLE 14
Variants with mutation at position 318 and at least
one further mutation (Combinatorial site mutagenesis)

| Met 171 | Glu 318 | FIOWT |
|---|---|---|
| M—Met | E—Glu | 1.0 |
| F—Phe | E—Glu | 2.1 |
| F—Phe | K—Lys | 5.0 |

Aqueous reactions contained 1% [w/v] X, one-time freeze-thawed cells (robot cultivated cells resuspended in 50 μL buffer, and 40 μL thereof used), 2M isopropylamine hydrochloride, 30% [v/v] DMSO and were incubated at 50° C. for 20 h. In all cases, ee (XI) > 99.5%.

TABLE 15
Variants with mutation at position 320 (Single-site mutagenesis)

| AA | FIOWT |
|---|---|
| N—Asn (wild-type) | 1.0 |
| L—Leu | 2.6 |
| K—Lys | 2.3 |
| C—Cys | 2.1 |
| M—Met | 1.9 |
| H—His | 1.9 |

Aqueous reactions contained 1% [w/v] X, one-time freeze-thawed cells (robot cultivated cells resuspended in 50 μL buffer, and 40 μL thereof used), 2M isopropylamine hydrochloride, 30% [v/v] DMSO and were incubated at 50° C. for 20 h. In all cases, ee (XI) > 99.5%.

TABLE 16
Variants with single mutation at position
320 and at least one further mutation
(Combinatorial site mutagenesis)

| Met | Ala | Trp | Met | Ile | Glu | Asn |
|---|---|---|---|---|---|---|
| 61 | 62 | 65 | 171 | 266 | 318 | 320 |
| L-Leu | A-Ala | M-Met | M-Met | V-Val | E-Glu | N-Asn |
| L-Leu | A-Ala | M-Met | M-Met | V-Val | K-Lys | M-Met |
| L-Leu | A-Ala | M-Met | M-Met | V-Val | E-Glu | N-Asn |
| L-Leu | S-Ser | M-Met | M-Met | V-Val | K-Lys | M-Met |
| | | Arg | His | Val | Ile | Conv. |
| | | 419 | 420 | 421 | 426 | [%] |
| | | P-Pro | H-His | V-Val | I-lle | 18.3 |
| | | P-Pro | N-Asn | S-Ser | I-lle | 51.5 |
| | | V-Val | H-His | V-Val | V-Val | 10.6 |
| | | V-Val | N-Asn | S-Ser | V-Val | 52.3 |

Organic reactions contained 1% [w/v] X, lyophilized cells (~17 mg CWW from 0.5 mL cultures in deep-well plates), water-saturated isopropyl acetate, 5 eq isopropylamine free base, and were incubated at 60° C. for 20 h. In all cases, ee (XI) > 99.5%.

TABLE 17
Variants with single mutation at position
419 (Single site mutagenesis)

| AA | FIOWT |
|---|---|
| R—Arg (wild-type) | 1.00 |
| L—Leu | 2.42 |
| A—Ala | 2.48 |
| C—Cys | 3.04 |
| V—Val | 3.05 |
| P—Pro | 3.92 |
| G—Gly | 4.20 |

Aqueous reactions contained 0.2% [w/v] X, 34% [v/v] lysate, 1M isopropylamine hydrochloride, 4% [v/v] DMSO and were incubated at 50° C. for 2 h. In all cases, ee (XI) > 99.5%.

TABLE 18
Variants with single mutation at position 419 and at least
one further mutation (Combinatorial site mutagenesis)

| Met 61 | Trp 65 | Ile 266 | Arg 419 | Ile 426 | FIOWT |
|---|---|---|---|---|---|
| M—Met | W—Trp | I—Ile | R—Arg | I—Ile | 1 |
| M—Met | W—Trp | I—Ile | P—Pro | I—Ile | 30 |
| M—Met | W—Trp | V—Val | P—Pro | L—Leu | 41 |
| M—Met | W—Trp | V—Val | P—Pro | V—Val | 44 |
| M—Met | V—Val | V—Val | A—Ala | V—Val | 57 |
| M—Met | C—Cys | I—Ile | A—Ala | I—Ile | 60 |
| L—Leu | M—Met | V—Val | C—Cys | L—Leu | 73 |
| L—Leu | V—Val | I—Ile | P—Pro | V—Val | 79 |
| L—Leu | V—Val | I—Ile | V—Val | V—Val | 79 |

Aqueous reactions contained 1% [w/v] X, one-time freeze-thawed cells (manually cultivated cells in 0.5 mL, OD600~10, resuspended in 200 μL buffer, 50 μL thereof used), 2M isopropylamine hydrochloride, 30% [v/v] DMSO, and were incubated at 50° C. for 20 h. In all cases, ee (XI) > 99.5%.

Variants with mutation at position 419 and other mutations on positions 61, 65, 266 and 426 (Combinatorial site mutagenesis): See Table 2, 3, 9 and 16.

TABLE 19
Variants with single mutation at position
420 (Single site mutagenesis)

| AA | FIOWT |
|---|---|
| H—His (wild-type) | 1.0 |
| D—Asp | 7.2 |
| N—Asn | 4.7 |

Aqueous reactions contained 1% [w/v] X, one-time freeze-thawed cells (robot cultivated cells resuspended in 50 μL buffer, and 40 μL thereof used), 2M isopropylamine hydrochloride, 30% [v/v] DMSO and were incubated at 50° C. for 20 h. In all cases, ee (XI) > 99.5%.

Variants with mutation at position 420 and at least one further mutation (Combinatorial site mutagenesis): See Table 2, 3, 9 and 16.

TABLE 20
Variants with single mutation at position
421 (Single site mutagenesis)

| AA | FIOWT |
|---|---|
| V—Val (wild-type) | 1.0 |
| S—Ser | 2.0 |

Aqueous reactions contained 1% [w/v] X, one-time freeze-thawed cells (robot cultivated cells resuspended in 50 μL buffer, and 40 μL thereof used), 2M isopropylamine hydrochloride, 30% [v/v] DMSO, and were incubated at 50° C. for 20 h. ee (XI) > 99.5%.

Variants with mutation at position 421 and at least one further mutation (Combinatorial site mutagenesis): See Table 2, 3, 9 and 16.

TABLE 21
Variants with single mutation at position
426 (Single site mutagenesis)

| AA | FIOWT |
|---|---|
| I—Ile (wild-type) | 1.00 |
| L—Leu | 1.24 |
| P—Pro | 1.61 |
| V—Val | 2.37 |

Aqueous reactions contained 0.2% [w/v] X, 34% [v/v] lysate, 1M isopropylamine hydrochloride, 4% [v/v] DMSO, and were incubated at 50° C. for 2 h. In all cases, ee (XI) > 99.5%.

Variants with mutation at position 426 and at least one further mutation (Combinatorial site mutagenesis): See Table 5, 7, 9, 11 and 16.

Variants with mutation at position 464 and at least one further mutation (Combinatorial site mutagenesis): See Table 11.

### EXAMPLE 3: AQUEOUS AND ORGANIC REACTIONS WITH SUBSTRATE X

3.1 1 ml MARS/1% [w/w] Substrate Loading:

The reaction mixture contained X (10 mg, 48.7 μmol, 1 eq.), solvent (1 ml), isopropylamine (20 μl, 0.2 mmol, 5 eq.), potassium phosphate buffer (30 μl, 1 M pH 7.2, contains 5 M isopropylamine hydrochloride and 2 mM PLP) and lyophilized whole cells mutant transaminase containing the mutant transaminase of SEQ ID NO. 3. The mixture was heated to 50° C. and shaken for a defined time (≥1d). The achieved product formation was determined by HPLC in area precent (a %). Results see Table 22.

TABLE 22
MARS process variables

| | Conversion |
|---|---|
| Organic Solvent | [a %] |
| THF, Me-THF, 1.2-Dimethoxyethan, Dimethoxymethan | 0.0 |
| TBME | 4.6 |
| Diethoxymethan | 2.7 |
| Toluene | 5.9 |
| Ethyl acetate | 43 |
| Isopropyl acetate | 63 |
| Isobutyl acetate | 51 |
| iso-propyl 2-methylpropanoate | 54 |
| tert-butyl 2,2-dimethylpropanoate | 54 |

The reaction mixture contained X (5 mg, 24.4 μmol, 1 eq.), isopropyl acetate (0.5 ml containing 2.0% H₂O), isopropylamine (10 μl, 0.2 mmol, 5 eq.) and lyophilized whole cells mutant transaminases (SEQ ID NO. 1-12). The mixture was heated to 60° C. and shaken for a defined time. The achieved product formation (XI) was determined by HPLC in area percent (a %). Results see Table 23.

TABLE 23
Comparison of mutant transaminase at 1% substrate loading

| | conversion | | conversion | |
|---|---|---|---|---|
| Mutant of | [a %], t = 4 h | FIOP | [a %], t = 24 h | FIOP |
| Blank (no enzyme) | 0 | 0 | 0 | 0 |
| SEQ ID NO: 1 | 0.6 | 1 | 1.1 | 1 |
| SEQ ID NO: 2 | 6.1 | 10 | 11.8 | 11 |
| SEQ ID NO: 3 | 9.1 | 14 | 32.2 | 31 |
| SEQ ID NO: 4 | 17.0 | 27 | 60.1 | 57 |
| SEQ ID NO: 5 | 14.6 | 23 | 56.2 | 53 |
| SEQ ID NO: 6 | 5.2 | 8 | 37.3 | 36 |
| SEQ ID NO: 7 | 12.1 | 19 | 44.0 | 42 |
| SEQ ID NO: 8 | 14.7 | 23 | 56.1 | 53 |
| SEQ ID NO: 9 | 8.7 | 14 | 47.7 | 45 |
| SEQ ID NO: 10 | 11.9 | 19 | 47.6 | 45 |
| SEQ ID NO: 11 | 5.4 | 8 | 20.1 | 19 |
| SEQ ID NO: 12 | 6.9 | 11 | 36.9 | 35 |

3.2 1 ml MARS/5% [w/w] Substrate Loading

The reaction mixture contained X (50 mg, 48.7 μmol, 1 eq.), isopropyl acetate (1 ml, water saturated 2.0% [w/w] resp. defined in Table 24), isopropylamine (100 μl, 1.2 mmol, 5 eq.), and lyophilized whole cells with mutant transaminase (25 mg, s/e 2). The mixture was heated to 60° C. resp. defined in Table 25) and shaken for the indicated time. The achieved product formation (XI) was determined by HPLC in area precent (a %).

TABLE 24
Influence of water content

| | Conc (H₂O) [% w/w] | Conversion |
|---|---|---|
| Mutant of | after reaction | [a %], t = 20 h |
| SEQ ID NO: 8 | 1.5 | 87 |
| SEQ ID NO: 8 | 1.6 | 86 |
| SEQ ID NO: 8 | 1.8 | 90 |
| SEQ ID NO: 8 | 2.1 | 89 |
| SEQ ID NO: 8 | 2.2 | 87 |

TABLE 25
Influence of temperature

| | | conversion |
|---|---|---|
| Mutant of | T [° C.] | [a %], t = 17 h |
| SEQ ID NO: 8 | 55 | 75.1 |
| SEQ ID NO: 8 | 60 | 81.6 |
| SEQ ID NO: 3 | 60 | 72.8 |
| SEQ ID NO: 8 | 65 | 82.4 |
| SEQ ID NO: 8 | 70 | 82.2 |

3.3 50 mL MARS/10% [w/w] Substrate Loading

The reaction contained X (5 g, 24.4 mmol, 1 eq), isopropyl acetate (30.6 g; 35 ml) containing 2.0% [w/w] water and lyophilized E. coli cells (2.5 g) containing the mutant transaminase of SEQ ID NO: 8. After stirring of the mixture for 5 min, the reaction was started by the addition of isopropylamine (6.9 g, 10 ml, 4.8 eq.), heated up to 60° C. and was allowed to stir for 24 h. The achieved product formation (XI) of 76.4 a % was determined by HPLC.

Subsequently the biocatalyst (lyophilized whole cells) was removed by filtration and washed with isopropyl acetate (20 ml). The combined filtrates have been evaporate to 20 g-removal of the remaining isopropylamine, water and the byproduct acetone. The concentrated product XI solution was diluted with isopropyl acetate (30 ml) and once more evaporated to 22 g and again diluted with isopropyl acetate (30 ml).

The product XI solution was heated to 60° C. and the product was precipitated as HCl salt by the addition of 5.8 M HCl isopropyl acetate solution (3.3 ml, 18.9 mmol, 0.8 eq.). The solution was aged under stirring at 60° C. for further 16 h. The crystals repeatedly isolated by filtration, washed with isopropyl acetate, digested first at 50° for 2 h in acetone (50 ml) and subsequently digested at 23° C. 16 h in acetone (50 ml) and finally dried using high vacuum.

3.7 g (68.2%) HCl salt as an off-white powder product XI.HCl was isolated with a HPLC purity of 99.6 a % XI.HCl (0.4 a % substrate X), NMR purity>95% (containing≤0.5% [w/w] isopropylamine, traces of acetone). Mass confirmed by LCMS.

3.4 1 L-Scale Reaction/5% [w/w] Substrate Loading

The reaction mixture was prepared by adding subsequently X (50 g, 243.7 mmol, 1 eq.), isopropyl acetate (500 g, 574.7 ml), isopropylamine (72 g. 104.7 ml, 1218.1 mmol, 5 eq.) and water (21.7 g, 2.6% [w/w]). The mixture was heated to 45° C. under stirring, prior to starting the reaction by the addition of a suspension of spray-dried E. coli cells (16.6 g) containing the mutant transaminase of SEQ ID NO: 8 in isopropyl acetate (225 g, 258.5 ml). The reaction temperature was heated up to 60° C. and was allowed to stir for additional 19 h. The achieved product formation was 84.5 a % (determined by HPLC).

Subsequently the biocatalyst (spray-dried whole cells) was removed by filtration and washed with isopropyl acetate (200 ml, 45° C.). The combined filtrates have been evaporate to 334 g—removal of the remaining isopropylamine, water and the byproduct acetone. The concentrated product XI solution was diluted with isopropyl acetate (800 ml).

The product XI solution was heated to 60° C. and the product was precipitated as HCl salt by the addition of 5.8 M HCl isopropyl acetate solution (42.0 ml, 243.7 mmol, ~1.2 eq.). The solution was aged under stirring at 60° C. for further 16 h. The crystals repeatedly isolated by filtration, washed with isopropyl acetate (400 ml) and acetone (250 ml) and subsequently dried using high vacuum.

50.4 g (83.8%) HCl salt as an off-white powder product XI.HCl was isolated with a HPLC purity of 98.3 a % XI.HCl (1.0 a % substrate X), NMR purity>95% (containing≤1.0% [w/w] isopropylamine, traces of acetone). Mass confirmed by LCMS.

3.5 2 L-Scale Reaction/5% [w/w] Substrate Loading/s/e 3

The reaction mixture was prepared by adding subsequently X (90.0 g, 192.5 mmol, 1 eq.), isopropyl acetate (875 g, 1006 ml), isopropylamine (129.6 g, 5 eq.) and water (31.1 g, 2.0% [w/w]). The mixture was stirred at 22° C., prior to starting the reaction by the addition of a suspension of spray-dried E. coli cells (30.0 g) containing mutant transaminase of SEQ ID NO: 8 (02219422AN0826) and PLP (0.6 g) in isopropyl acetate (386 g, 444 ml). The reaction temperature was heated up to 60° C. and was allowed to stir for additional 8 h. The achieved product formation was roughly 80 a % (determined by HPLC). The reaction volume was halved by distilling—removal of the byproduct acetone. The reaction mixture was replenished with isopropyl acetate (652 g, 750 ml), isopropylamine (129.6 g, 5 eq.) and water (30 g) and allowed to complete for additional 16 h achieving a product formation of 91.5%.

Subsequently Dicalite (15 g) was added, stirred (45 min.) prior to filtering of the biocatalyst (spray-dried whole cells). The biocatalyst is washed with isopropyl acetate (345 ml) at 45° C. The combined filtrates have been concentrated by evaporation to 180 ml (2 V, removal of the remaining isopropylamine, water and acetone).

The concentrated product XI solution was diluted with isopropyl acetate (450 ml, 5 V) and water (450 ml, 5 V) and heated to 50° C. The pH of the mixture was adjusted to be below 1 by the addition of aqueous HCl solution (47.5 g, 0.95 eq.). The mixture was heated to 65° C. and the phases were separated. The organic phase was once more extracted with water (180 ml, 2 V). The aqueous solutions were combined and the solvent was changed to isopropanol by aceotropic water removal keeping the volume constant (450 ml, 5V). The temperature was reduced to 0° C. in order to completely crystalize product XI. After filtering off product XI, it was washed with isopropanol (180 ml, 2 V) at 0° C. and subsequently dried using high vacuum.

94.7 g (89.0%) HCl salt as an off-white crystalline product XI.HCl was isolated with a HPLC purity of 99.9 a % XI.HCl (0.05 a % substrate X, ≤0.1% isopropanol).

3.6 0.8 L-Scale Reaction/5% [w/w] Substrate Loading/s/e 4

The reaction mixture was prepared by adding subsequently X (39.5 g, 192.5 mmol, 1 eq.), isopropyl acetate (385 g, 442 ml), isopropylamine (56.7 g, 5 eq.) and water (13.6 g, 2.0% [w/w]). The mixture was stirred at 22° C., prior to starting the reaction by the addition of a suspension of spray-dried E. coli cells (10.0 g) containing mutant transaminase of SEQ ID NO: 8 in isopropyl acetate (170 g, 195 ml). The reaction temperature was heated up to 60° C. and was allowed to stir for additional 8 h. The achieved product formation was roughly 80 a % (determined by HPLC). The reaction volume was halved by distilling-removal of the byproduct acetone. The reaction mixture was replenished with isopropyl acetate (278 g, 320 ml), isopropylamine (56.7 g, 5 eq.) and water (13.1 g) and allowed to complete for additional 16 h achieving a product formation of 91.5%.

Subsequently Dicalite (5 g) was added, stirred (45 min.) prior to filtering of the biocatalyst (spray-dried whole cells). The biocatalyst is washed with isopropyl acetate (150 ml, 45° C.). The combined filtrates have been concentrated by evaporation to 80 ml (2 V, removal of the remaining isopropylamine, water and acetone.

The concentrated product XI solution was diluted with isopropyl acetate (200 ml, 5 V) and water (200 ml, 5 V) and heated to 50° C. The pH of the mixture was adjusted to be below 1 by the addition of aqueous HCl solution (23 g, 1.05 eq.). The mixture was heated to 65° C. and the phases were separated. The organic phase was once more extracted with water (80 ml, 2 V). The aqueous solutions were combined and the solvent was changed to isopropanol by aceotropic water removal keeping the volume constant (200 ml, 5V). The temperature was reduced to 0° C. in order to completely crystalize product XI. After filtering off product XI, it was washed with isopropanol (80 ml) at 0° C. and subsequently dried using high vacuum.

38.3 g (82.0%) HCl salt as an off-white crystalline product XI.HCl was isolated with a HPLC purity of 99.8 a % XI.HCl (0.1 a % substrate X, ≤0.1% [w/w] isopropylamine, water and/or isopropanol).

### EXAMPLE 4: AQUEOUS AND ORGANIC REACTIONS WITH OTHER SUBSTRATES

### 4.1 Selection of Commercially Available Ketones

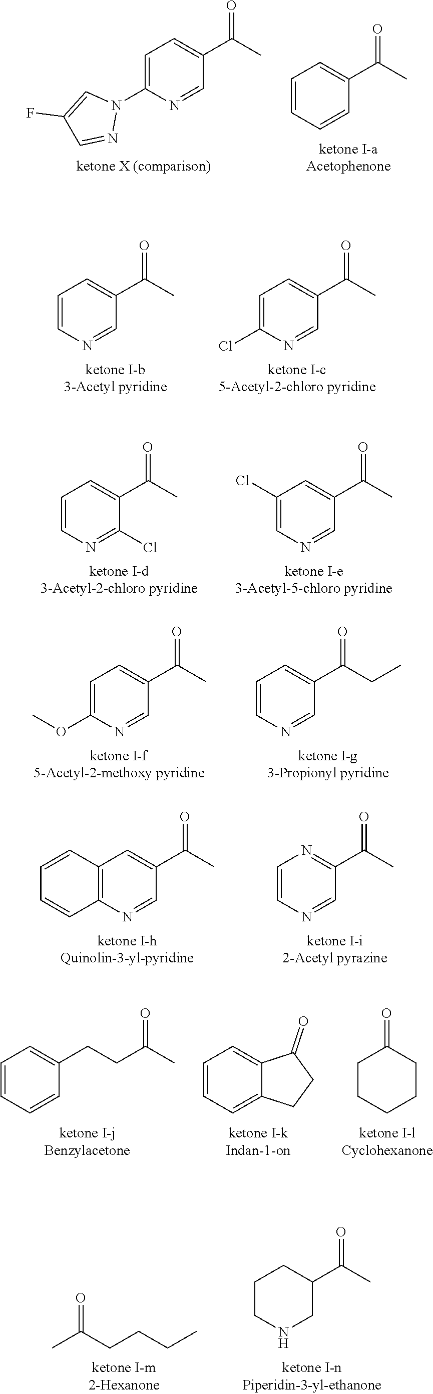

### 4.2 General Enzymatic Amine Synthesis on Small Scale at 1% Substrate Loading

The reaction mixture contained a ketone (5 mg, 1 eq., I-a to I-n and X, isopropyl acetate (0.5 ml containing 2.0% H₂O), isopropylamine (10 μl, 0.2 mmol, 5 eq.) and lyophilized mutant transaminase SEQ ID NO. 8 (1.67 mg; s/e 3). The mixture was heated to 60° C. and shaken for 24 h. The achieved product formation II and its enantiomeric excess were determined by corresponding chromatographic methods in area percent (a %) and percent (%).

TABLE 26
Product formations at 1% loading of various substrates

| | 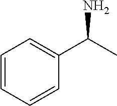 | 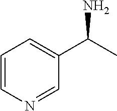 | 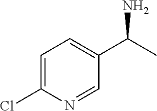 | 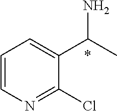 | 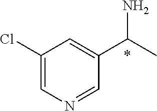 |
|---|---|---|---|---|---|
| Product | (S)-II-a | (S)-II-b | (S)-II-c | II-d | II-e |
| conv. | 70 | 90 | 94 | 98^{a} | 97 |
| [a %] | | | | | |
| ee [%] | 99.9 | 99.9 | 99.9 | 99.9 | 99.9 |
| | 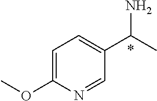 | 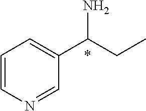 | 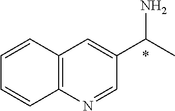 | 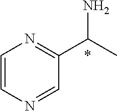 | 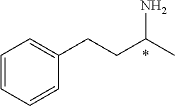 |
| Product | II-f | II-g | II-h | II-i | II-j |
| conv. | 65 | 77^{b} | 87 | 99 | 75 |
| [a %] | | | | | |
| ee [%] | 99.9 | 99.9 | 99.9 | n.d.^{c} | 99.9 |
| | 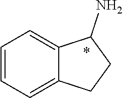 | 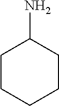 | 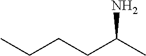 | 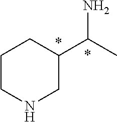 | 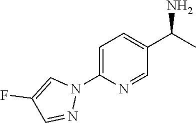 |
| Product | II-k | II-l | (S)-II-m | II-n | (S)-XI |
| conv. | 27 | 98 | 73 | 72 | 88 |
| [a %] | | | | | |
| ee [%] | 99.9 | n.a.^{d} | 99.9 | —^{e} | 99.9 |

^{a}(5 mg; s/e 1.0);
^{b}(15 mg; s/e 0.3);
^{c}product not isolated;
^{d}not chiral;
^{e}diastereomeric ratio not determined

1. A mutant transaminase with increased asymmetric reductive amination activity relative to the wild-type transaminase,
wherein the mutant transaminase comprises an amino acid sequence that is at least 80% identical to the amino acid sequence of SEQ ID NO: 1 (transaminase from Ruegeria pomeroyi),
wherein the mutant transaminase has at least four amino acid substitutions at positions 61, 65, 266 and 419 relative to the amino acid sequence of SEQ ID NO: 1, and
wherein at least three of the substitutions are selected from the group consisting of:
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266), and
the amino acid at the position corresponding to position 419 of SEQ ID NO: 1 is substituted with Pro (Pro419) or Val (Val419).

2. The mutant transaminase of claim 1, wherein
the amino acid at the position corresponding to position 61 of SEQ ID NO: 1 is substituted with Leu (Leu61);
the amino acid at the position corresponding to position 65 of SEQ ID NO: 1 is substituted with Met (Met65);
the amino acid at the position corresponding to position 266 of SEQ ID NO: 1 is substituted with Val (Val266), and
the amino acid at the position corresponding to position 419 of SEQ ID NO: 1 is substituted with Pro (Pro419) or Val (Val419).

3. The mutant transaminase of claim 1, wherein
the amino acid at the position corresponding to position 426 of SEQ ID NO: is substituted with Val (Val426).

4. The mutant transaminase of claim 1, wherein
the amino acid at the position corresponding to position 61 of SEQ ID NO: 1 is substituted with Leu (Leu61); and
the amino acid at the position corresponding to position 65 of SEQ ID NO: 1 is substituted with Met (Met65); and
the amino acid at the position corresponding to position 266 of SEQ ID NO: 1 is substituted with Val (Val266); and
the amino acid at the position corresponding to position 419 of SEQ ID NO: 1 is substituted with Pro (Pro419); or
wherein
the amino acid at the position corresponding to position 61 of SEQ ID NO: 1 is substituted with Leu (Leu61); and
the amino acid at the position corresponding to position 65 of SEQ ID NO: 1 is substituted with Met (Met65); and
the amino acid at the position corresponding to position 266 of SEQ ID NO: 1 is substituted with Val (Val266); and
the amino acid at the position corresponding to position 419 of SEQ ID NO: 1 is substituted with Val (Val419); and
the amino acid at the position corresponding to position 426 of SEQ ID NO: 1 is substituted with Val (Val426).

5. The mutant transaminase of claim 1, wherein
the amino acid at the position corresponding to position 9 of SEQ ID NO: 1 is substituted with Tyr (Tyr9); and/or
the amino acid at the position corresponding to position 62 of SEQ ID NO: 1 is substituted with Ser (Ser62); and/or
the amino acid at the position corresponding to position 65 of SEQ ID NO: 1 is substituted with Phe (Phe65), Gly (Gly65) or Met (Met65); and/or
the amino acid at the position corresponding to position 171 of SEQ ID NO: 1 is substituted with Phe (Phe171) or Trp (Trp171), and/or
the amino acid at the position corresponding to position 198 of SEQ ID NO: 1 is substituted with Leu (Leu198) or Met (Met198); and/or
the amino acid at the position corresponding to position 318 of SEQ ID NO: 1 is substituted with Lys (Lys318), and/or
the amino acid at the position corresponding to position 320 of SEQ ID NO: 1 is substituted with Cys (Cys320), Leu (Leu318), Lys (Lys320), or Met (Met320), and/or
the amino acid at the position corresponding to position 419 of SEQ ID NO: 1 is substituted with Ala (Ala419), Cys (Cys419), Gly (Gly419), Pro (Pro419) or Val (Val419), and/or
the amino acid at the position corresponding to position 420 of SEQ ID NO: 1 is substituted with Asn (Asn420), Asp (Asp420), Cys (Cys420) or Ser (Ser420), and/or
the amino acid at the position corresponding to position 421 of SEQ ID NO: 1 is substituted with Ser (Ser421), and/or
the amino acid at the position corresponding to position 464 of SEQ ID NO: is substituted with Met (Met464).

6. The mutant transaminase of claim 1, wherein
the mutant transaminase has the substitutions Leu61, Met65, Val266 and Pro419; or
the mutant transaminase has the substitutions Leu61, Met65, Val266, Val419 and Val426; or
the mutant transaminase has the substitutions Leu61, Ser62, Met65, Phe171, Val266, Lys318, Pro419, Asn420 and Ser421; or
the mutant transaminase has the substitutions Leu61, Ser62, Met65, Phe171, Val266, Lys318, Met320, Pro419, Asn420 and Ser421; or
the mutant transaminase has the substitutions Leu61, Met65, Phe171, Val266, Cys320, Val419, Asn420, Ser421 and Val426; or
the mutant transaminase has the substitutions Tyr9, Leu61, Ser62, Met65, Phe171, Val266, Lys318, Pro419, Asn420 and Ser421; or
the mutant transaminase has the substitutions Tyr9, Leu61, Ser62, Met65, Phe171, Val266, Lys318, Met320, Pro419, Asn420 and Ser421; or
the mutant transaminase has the substitutions Tyr9, Leu61, Met65, Phe171, Val266, Cys320, Val419, Asn420, Ser421 and Val426; or
the mutant transaminase has the substitutions Leu61, Ser62, Met65, Phe171, Asp198, Val266, Lys318, Pro419, Asn420, Ser421 and Met464; or
the mutant transaminase has the substitutions Leu61, Ser62, Met65, Phe171, Asp198, Val266, Lys318, Met320, Pro419, Asn420, Ser421 and Met464; or
the mutant transaminase has the substitutions Leu61, Met65, Phe171, Asp198, Val266, Cys320, Val419, Asn420, Ser421, Val426 and Met464.

7. The mutant transaminase of claim 1, wherein
the mutant transaminase has the substitutions Leu61, Met65, Val266 and Pro419 and wherein the amino acids at positions corresponding to positions 9, 62, 171, 198, 318, 320, 420, 421, 426 and 464 of SEQ ID No: 1 are unsubstituted;
the mutant transaminase has the substitutions Leu61, Met65, Val266, Val419 and Val426 and wherein the amino acids at positions corresponding to positions 9, 62, 171, 198, 318, 320, 420, 421 and 464 of SEQ ID No:1 are unsubstituted; or
the mutant transaminase has the substitutions Leu61, Ser62, Met65, Phe171, Val266, Lys318, Pro419, Asn420 and Ser421 and wherein the amino acids at positions corresponding to positions 9, 198, 320, 426 and 464 of SEQ ID No:1 are unsubstituted; or
the mutant transaminase has the substitutions Leu61, Ser62, Met65, Phe171, Val266, Lys318, Met320, Pro419, Asn420 and Ser421 and wherein the amino acids at positions corresponding to positions 9, 198, 426 and 464 of SEQ ID No:1 are unsubstituted; or
the mutant transaminase has the substitutions Leu61, Met65, Phe171, Val266, Cys320, Val419, Asn420, Ser421 and Val426 and wherein the amino acids at positions corresponding to positions 9, 62, 198, 318, and 464 of SEQ ID No: 1 are unsubstituted; or
the mutant transaminase has the substitutions Tyr9, Leu61, Ser62, Met65, Phe171, Val266, Lys318, Pro419, Asn420 and Ser421 and wherein the amino acids at positions corresponding to positions 198, 320, 426 and 464 of SEQ ID No:1 are unsubstituted; or
the mutant transaminase has the substitutions Tyr9, Leu61, Ser62, Met65, Phe171, Val266, Lys318, Met320, Pro419, Asn420 and Ser421 and wherein the amino acids at positions corresponding to positions 198, 426 and 464 of SEQ ID No:1 are unsubstituted; or
the mutant transaminase has the substitutions Tyr9, Leu61, Met65, Phe171, Val266, Cys320, Val419, Asn420, Ser421 and Val426 and wherein the amino acids at positions corresponding to positions 62, 198, 318, and 464 of SEQ ID No: 1 are unsubstituted; or
the mutant transaminase has the substitutions Leu61, Ser62, Met65, Phe171, Asp198, Val266, Lys318, Pro419, Asn420, Ser421 and Met464 and wherein the amino acids at positions corresponding to positions 9, 320, and 426 of SEQ ID No:1 are unsubstituted; or
the mutant transaminase has the substitutions Leu61, Ser62, Met65, Phe171, Asp198, Val266, Lys318, Met320, Pro419, Asn420, Ser421 and Met464 and wherein the amino acids at positions corresponding to positions 9 and 426 of SEQ ID No:1 are unsubstituted; or
the mutant transaminase has the substitutions Leu61, Met65, Phe171, Asp198, Val266, Cys320, Val419, Asn420, Ser421, Val426 and Met464 wherein the amino acids at positions corresponding to positions 9, 62, and 318 of SEQ ID No:1 are unsubstituted.

8. The mutant transaminase of claim 1, wherein
the mutant transaminase has the substitutions Leu61, Met65, Val 266 and Pro419; or
the mutant transaminase has the substitutions Leu61, Met65, Val266, Val419 and Val426; or
the mutant transaminase has the substitutions Leu61, Ser62, Met65, Phe171, Val 266, Lys318, Pro419, Asn420 and Ser421; or
the mutant transaminase has the substitutions Leu61, Ser62, Met65, Phe171, Val266, Lys318, Met320, Pro419, Asn420 and Ser421; or
the mutant transaminase has the substitutions Leu61, Met65, Phe171, Val266, Cys320, Val419, Asn420, Ser421 and Val426; or
the mutant transaminase has the substitutions Tyr9, Leu61, Ser62, Met65, Phe171, Val266, Lys318, Pro419, Asn420 and Ser421; or
the mutant transaminase has the substitutions Tyr9, Leu61, Ser62, Met65, Phe171, Val266, Lys318, Met320, Pro419, Asn420 and Ser421; or
the mutant transaminase has the substitutions Tyr9, Leu61, Met65, Phe171, Val266, Cys320, Val419, Asn420, Ser421 and Val426; or
the mutant transaminase has the substitutions Leu61, Ser62, Met65, Phe171, Asp198, Val266, Lys318, Pro419, Asn420, Ser421 and Met464; or
the mutant transaminase has the substitutions Leu61, Ser62, Met65, Phe171, Asp198, Val266, Lys318, Met320, Pro419, Asn420, Ser421 and Met464; or
the mutant transaminase has the substitutions Leu61, Met65, Phe171, Asp198, Val266, Cys320, Val419, Asn420, Ser421, Val426 and Met464,
wherein the defined substitutions are the only substitutions in the mutant transaminases relative to the amino acid sequence of SEQ ID NO: 1.

9. The mutant transaminase of claim 1, wherein the mutant transaminase consists of or comprises an amino acid sequence that is at least 85%, 90%, 95%, 96%, 97%, 98%, or 99%, or 100%, identical to the amino acid sequence of any of SEQ ID NO: 2 to 12.

10. The mutant transaminase of claim 1, wherein
the mutant transaminase has an increased reductive amination activity relative to the wild-type transaminase in the presence of an organic solvent and an amine donor; and/or
the mutant transaminase has increased stability in aqueous organic media; and/or
the mutant transaminase has an increased stability in the presence of an organic solvent and a residual amount of water; and/or
the mutant transaminase is capable of reductive amination of a ketone into a primary amine; and/or
the mutant transaminase shows reductive amination activity at a temperature of between 40° C. and 70° C.

11. The mutant transaminase of claim 1, wherein the mutant transaminase is capable of reductive amination of a ketone into a primary amine and wherein the ketone has the formula I 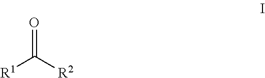
wherein
R¹ and R² independently of each other represent optionally substituted alkyl, aryl, carbocyclyl or heterocyclyl; or
R¹ and R² together with the carbon atom they are attached to form an optionally substituted mono- or poly-cyclic carbocyclic or heterocyclic ring, and
wherein optional substituents are selected from alkyl, alkoxy, aryl, heteroaryl, aryloxy, halogen, hydroxyl or cyano.

12. The mutant transaminase of claim 1, wherein the mutant transaminase has the potential to convert the ketone of formula I into the primary amine of formula II 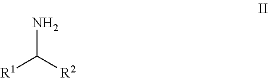
wherein
R¹ and R² independently of each other represent optionally substituted alkyl, aryl, carbocyclyl or heterocyclyl; or
R¹ and R² together with the carbon atom they are attached to form an optionally substituted mono- or poly-cyclic carbocyclic or heterocyclic ring, and
wherein optional substituents are selected from alkyl, alkoxy, aryl, heteroaryl, aryloxy, halogen, hydroxyl or cyano.

13. A nucleic acid coding for the mutant transaminase of claim 1, optionally comprised in a vector.

14. A cell comprising the mutant transaminase of claim 1.

15. A method for the enzymatic reductive transamination of a ketone and the formation of a primary amine in the presence of mutant transaminase of claim 1.

16. The method of claim 15, wherein the ketone is a prochiral ketone and the primary amine is a chiral primary amine.

17. The method of claim 15, wherein the ketone has the formula I 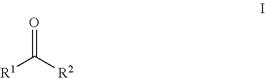
wherein
R¹ and R² independently of each other represent optionally substituted alkyl, aryl, carbocyclyl or heterocyclyl; or
R¹ and R² together with the carbon atom they are attached to form an optionally substituted mono- or poly-cyclic carbocyclic or heterocyclic ring, and
wherein optional substituents are selected from alkyl, alkoxy, aryl, heteroaryl, aryloxy, halogen, hydroxyl or cyano
and the resulting primary amine has the formula II 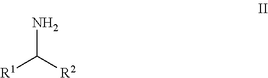
wherein
R¹ and R² independently of each other represent optionally substituted alkyl, aryl, carbocyclyl or heterocyclyl; or
R¹ and R² together with the carbon atom they are attached to form an optionally substituted mono- or poly-cyclic carbocyclic or heterocyclic ring, and
wherein optional substituents are selected from alkyl, alkoxy, aryl, heteroaryl, aryloxy, halogen, hydroxyl or cyano.

18. The method of claim 15, wherein enzymatic reductive transamination takes place in the presence of an organic solvent, which contains 1% wt. to 5 wt. % water.

19. The method of claim 18, wherein the organic solvent is a lower alkyl ester of acetic acid or propionic acid.

20. The method of claim 15, wherein the enzymatic reductive transamination takes place in the presence of an amine donor.

21. The method of claim 20, wherein the amine donor is a primary aliphatic amine.

22. The method of claim 20, wherein the amine donor is used in amounts of 2 eq. to 20 eq.

23. The method of claim 15, wherein the mutant transaminase is applied as enzyme immobilisate or as whole E. coli cells.

24. The method of claim 15, wherein enzymatic reductive transamination takes place between 40° C. and 70° C.

25. The method of claim 15, wherein the ketone of formula I is loaded in an amount of 1% wt. and 15% wt.

26. The mutant transaminase of claim 10, wherein
the aqueous media contains 4.0% v/v to 40.0% v/v of an organic solvent; and/or
the ketone is a prochiral ketone and the primary amine is a chiral primary amine; and/or
the mutant transaminase shows reductive amination activity at a temperature of between 50° C. and 70° C.


SEQUENCE LISTING (12 sequences)

SEQ ID NO: 1 - protein, 466 aa, Ruegeria pomeroyi
MSLATITNHMPTAELQALDAAHHLHPFSANNALGEEGTRVITRARGVWLNDSEGEEILDAMAGLWCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGMAGMHAQSGLIPDVHHINQPNWWAEGGDMDPEEFGLARARELEEAILELGENRVAAFIAEPVQGAGGVIVAPDSYWPEIQRICDKYDILLIADEVICGFGRTGNWFGTQTMGIRPHIMTIAKGLSSGYAPIGGSIVCDEVAHVIGKDEFNHGYTYSGHPVAAAVALENLRILEEENILDHVRNVAAPYLKEKWEALTDHPLVGEAKIVGMMASIALTPNKASRAKFASEPGTIGYICRERCFANNLIMRHVGDRMIISPPLVITPAEIDEMFVRIRKSLDEAQAEIEKQGLMKSAA

SEQ ID NO: 2 - protein, 466 aa, synthetic construct
MSLATITNHMPTAELQALDAAHHLHPFSANNALGEEGTRVITRARGVWLNDSEGEEILDALAGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGMAGMHAQSGLIPDVHHINQPNWWAEGGDMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDEFNHGYTYSGHPVAAAVALENLRILEEENILDHVRNVAAPYLKEKWEALTDHPLVGEAKIVGMMASIALTPNKASRAKFASEPGTIGYICRERCFANNLIMPHVGDRMIISPPLVITPAEIDEMFVRIRKSLDEAQAEIEKQGLMKSAA

SEQ ID NO: 3 - protein, 466 aa, synthetic construct
MSLATITNHMPTAELQALDAAHHLHPFSANNALGEEGTRVITRARGVWLNDSEGEEILDALAGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGMAGMHAQSGLIPDVHHINQPNWWAEGGDMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDEFNHGYTYSGHPVAAAVALENLRILEEENILDHVRNVAAPYLKEKWEALTDHPLVGEAKIVGMMASIALTPNKASRAKFASEPGTIGYICRERCFANNLIMVHVGDRMVISPPLVITPAEIDEMFVRIRKSLDEAQAEIEKQGLMKSAA

SEQ ID NO: 4 - protein, 466 aa, synthetic construct
MSLATITNHMPTAELQALDAAHHLHPFSANNALGEEGTRVITRARGVWLNDSEGEEILDALSGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGDMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDKFNHGYTYSGHPVAAAVALENLRILEEENILDHVRNVAAPYLKEKWEALTDHPLVGEAKIVGMMASIALTPNKASRAKFASEPGTIGYICRERCFANNLIMPNSGDRMIISPPLVITPAEIDEMFVRIRKSLDEAQAEIEKQGLMKSAA

SEQ ID NO: 5 - protein, 466 aa, synthetic construct
MSLATITNHMPTAELQALDAAHHLHPFSANNALGEEGTRVITRARGVWLNDSEGEEILDALSGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGDMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDKFMHGYTYSGHPVAAAVALENLRILEEENILDHVRNVAAPYLKEKWEALTDHPLVGEAKIVGMMASIALTPNKASRAKFASEPGTIGYICRERCFANNLIMPNSGDRMIISPPLVITPAEIDEMFVRIRKSLDEAQAEIEKQGLMKSAA

SEQ ID NO: 6 - protein, 466 aa, synthetic construct
MSLATITNHMPTAELQALDAAHHLHPFSANNALGEEGTRVITRARGVWLNDSEGEEILDALAGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGDMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDEFCHGYTYSGHPVAAAVALENLRILEEENILDHVRNVAAPYLKEKWEALTDHPLVGEAKIVGMMASIALTPNKASRAKFASEPGTIGYICRERCFANNLIMVNSGDRMVISPPLVITPAEIDEMFVRIRKSLDEAQAEIEKQGLMKSAA

SEQ ID NO: 7 - protein, 466 aa, synthetic construct
MSLATITNYMPTAELQALDAAHHLHPFSANNALGEEGTRVITRARGVWLNDSEGEEILDALSGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGDMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDKFNHGYTYSGHPVAAAVALENLRILEEENILDHVRNVAAPYLKEKWEALTDHPLVGEAKIVGMMASIALTPNKASRAKFASEPGTIGYICRERCFANNLIMPNSGDRMIISPPLVITPAEIDEMFVRIRKSLDEAQAEIEKQGLMKSAA

SEQ ID NO: 8 - protein, 466 aa, synthetic construct
MSLATITNYMPTAELQALDAAHHLHPFSANNALGEEGTRVITRARGVWLNDSEGEEILDALSGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGDMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDKFMHGYTYSGHPVAAAVALENLRILEEENILDHVRNVAAPYLKEKWEALTDHPLVGEAKIVGMMASIALTPNKASRAKFASEPGTIGYICRERCFANNLIMPNSGDRMIISPPLVITPAEIDEMFVRIRKSLDEAQAEIEKQGLMKSAA

SEQ ID NO: 9 - protein, 466 aa, synthetic construct
MSLATITNYMPTAELQALDAAHHLHPFSANNALGEEGTRVITRARGVWLNDSEGEEILDALAGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGDMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDEFCHGYTYSGHPVAAAVALENLRILEEENILDHVRNVAAPYLKEKWEALTDHPLVGEAKIVGMMASIALTPNKASRAKFASEPGTIGYICRERCFANNLIMVNSGDRMVISPPLVITPAEIDEMFVRIRKSLDEAQAEIEKQGLMKSAA

SEQ ID NO: 10 - protein, 466 aa, synthetic construct
MSLATITNHMPTAELQALDAAHHLHPFSANNALGEEGTRVITRARGVWLNDSEGEEILDALSGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGLMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDKFNHGYTYSGHPVAAAVALENLRILEEENILDHVRNVAAPYLKEKWEALTDHPLVGEAKIVGMMASIALTPNKASRAKFASEPGTIGYICRERCFANNLIMPNSGDRMIISPPLVITPAEIDEMFVRIRKSLDEAQAEIEKQGLMKMAA

SEQ ID NO: 11 - protein, 466 aa, synthetic construct
MSLATITNHMPTAELQALDAAHHLHPFSANNALGEEGTRVITRARGVWLNDSEGEEILDALSGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGLMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDKFMHGYTYSGHPVAAAVALENLRILEEENILDHVRNVAAPYLKEKWEALTDHPLVGEAKIVGMMASIALTPNKASRAKFASEPGTIGYICRERCFANNLIMPNSGDRMIISPPLVITPAEIDEMFVRIRKSLDEAQAEIEKQGLMKMAA

SEQ ID NO: 12 - protein, 466 aa, synthetic construct
MSLATITNHMPTAELQALDAAHHLHPFSANNALGEEGTRVITRARGVWLNDSEGEEILDALAGLMCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGFAGMHAQSGLIPDVHHINQPNWWAEGGLMDPEEFGLARARELEEAILELGENRVAAFIAEPVQGAGGVIVAPDSYWPEIQRICDKYDILLIADEVVCGFGRTGNWFGTQTMGIRPHIMTIAKGLSSGYAPIGGSIVCDEVAHVIGKDEFCHGYTYSGHPVAAAVALENLRILEEENILDHVRNVAAPYLKEKWEALTDHPLVGEAKIVGMMASIALTPNKASRAKFASEPGTIGYICRERCFANNLIMVNSGDRMVISPPLVITPAEIDEMFVRIRKSLDEAQAEIEKQGLMKMAA